(19) United States
(12) Patent Application Publication
(10) Pub. No.: US 2010/0041696 A1
(43) Pub. Date: Feb. 18, 2010

(54) Compounds

(76) Inventors: Alain Claude-Marie Daugan, Les Ulis (FR); Anthony William Dean, Hertfordshire (GB)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 12/519,877

(22) PCT Filed: Dec. 19, 2007
(86) PCT No.: PCT/EP2007/064192

§ 371 Date: Jun. 18, 2009

(30) Foreign Application Priority Data
Dec. 21, 2006 (GB) 0625594.7

(51) Int. Cl. A61K 31/47; C07D 401/12

(52) U.S. Cl. 514/310; 546/143

(57) ABSTRACT

The present invention relates to substituted 4-Aminopyrazole compounds of the formula (I):

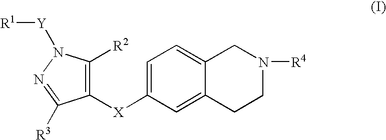

and pharmaceutically acceptable salts thereof, to pharmaceutical compositions containing them and their use in medicine. In particular, the invention relates to compounds for modulating SCD activity.

### FIELD OF THE INVENTION

The present invention relates to a novel class of compounds believed to be inhibitors of stearoyl-CoA desaturase (SCD), compositions comprising said compounds, methods of synthesis and uses for such compounds in treating and/or preventing various diseases, including those mediated by SCD enzyme, such as diseases related to elevated lipid levels, cardiovascular disease, diabetes, obesity, metabolic syndrome, skin disorders such as acne, diseases or conditions related to cancer and the treatment of symptoms linked to the production of the amyloid plaque-forming Aβ42 peptide such as Alzheimer's disease and the like.

### BACKGROUND OF THE INVENTION

Acyl desaturase enzymes catalyze the formation of double bonds in fatty acids derived from either dietary sources or de novo synthesis in the liver. Mammals synthesise at least three fatty acid desaturases of differing chain length that specifically catalyze the addition of double bonds at the delta-9, delta-6, and delta-5 positions. Stearoyl-CoA desaturases (SCDs) introduce a double bond in the C₉-C₁₀ position of saturated fatty acids. The preferred substrates for the enzymes are palmitoyl-CoA (16:0) and stearoyl-CoA (18:0), which are converted to palmitoleoyl-CoA (16:1) and oleoyl-CoA (18:1), respectively. The resulting mono-unsaturated fatty acids may then be employed in the preparation of phospholipids, triglycerides, and cholesteryl esters, in vivo.

A number of mammalian SCD genes have been cloned. For example, two genes have been cloned from rats (SCD1, SCD2) and four SCD genes have been isolated from mice (SCD1, 2, 3 and 4). While the basic biochemical roles of SCD has been known in rats and mice since the 1970's (Jeffcoat, R et al., Elsevier Science (1984), Vol 4, pp. 85-112; de Antueno, R J, Lipids (1993), Vol. 28, No. 4, pp. 285-290), it has only recently been directly implicated in human diseases processes.

A single SCD gene, SCD1, has been characterized in humans. SCD1 is described in Brownlie et al, WO 01/62954. A second human SCD isoform has been identified, and because it bears little sequence homology to known mouse or rat isoforms it has been named human SCD5 or hSCD5 (WO 02/26944).

Whilst not wishing to be bound by theory, it is thought that inhibition of the activity of SCD in vivo can be used to ameliorate and/or treat one or more diseases such as dyslipidemia, hypoalphalipoproteinemia, hyperbetalipoproteinemia, hypercholesterolemia, hypertriglyceridemia, familial hypercholesterolemia, angina, ischemia, cardiac ischemia, stroke, myocardial infarction, atherosclerosis, obesity, Type I diabetes, Type II diabetes, insulin resistance, hyperinsulinaemia, metabolic syndrome; other cardiovascular diseases e.g. peripheral vascular disease, reperfusion injury, angioplastic restenosis, hypertension, vascular complications of diabetes, thrombosis; hepatic steatosis, non-alcoholic steatohepatitis (NASH) and other diseases related to accumulation of lipids in the liver.

An SCD-mediated disease or condition also includes a disorder of polyunsaturated fatty acid (PUFA) disorder, or a skin disorder, including but not limited to eczema, acne, psoriasis, keloid scar formation or prevention, diseases related to production or secretions from mucous membranes, such as monounsaturated fatty acids, wax esters, and the like (US2006/0205713A1, WO2007/046868, WO2007/046867). SCD has been shown to play a physiological role in cholesterol homeostasis and the de novo biosynthesis of cholesterol esters, triglycerides and wax esters required for normal skin and eyelid function and therefore may be useful in the treatment of acne and other skin conditions (Makoto et al. J of Nutrition (2001), 131(9), 2260-2268, Harrison et al. J of Investigative Dermatology (2007) 127(6), 1309-1317).

An SCD-mediated disease or condition also includes but is not limited to a disease or condition which is, or is related to cancer, neoplasia, malignancy, metastases, tumours (benign or malignant), carcinogenesis, hepatomas and the like (US2006/0205713A1, WO2007/046868, WO2007/046867). Recently, SCD-1 has been identified as playing a role in human tumor cell survival and therefore has potential as an anticancer target (Morgan-Lappe et al. 2007 Cancer Res. 67 (9) 4390-4398).

It has been shown that overexpression of Steroyl-CoA desaturase (SCD) in human cells in culture leads to a specific increase in the production of the amyloid plaque-forming Aβ42 peptide, and conversely, that reductions in SCD activity in human cells in culture leads to a specific decrease in the production of Aβ42. Therefore, SCD inhibitors may also be useful for treating, delaying the onset of symptoms, or slowing the progression of symptoms of mild cognitive impairment (MCI), Alzheimer's Disease (AD), cerebral amyloid angiopathy (CAA) or dementia associated with Down Syndrome (DS) and other neurodegenerative diseases characterized by the formation or accumulation of amyloid plaques comprising Aβ42 (US2007/0087363A1; Myriad Genetics).

WO2005/011657 describes certain piperazine derivatives useful for inhibiting SCD activity.

The present invention provides a compound of formula (I) for inhibiting SCD activity:

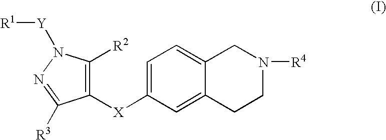

wherein:
X represents —CONH—, —NHCO— or —NHCONH—,
R¹ represents:
(i) a substituent selected from: H, —C₁₋₆alkyl or —C₃₋₆cycloalkyl,
(ii) —C₆₋₁₀aryl (such as phenyl or naphthyl) optionally substituted by one, two or three groups independently selected from:
(a) —C₁₋₆alkyl (such as —CH₃), —C₁₋₆haloalkyl (such as —CF₃), —C₃₋₆cycloalkyl, —C₁₋₆alkoxy (such as —OCH₃), —OR⁵, —CN or halogen (such as chloro, bromo or fluoro),
(b) —C₆₋₁₀aryl (such as phenyl), —C₅₋₁₀heteroaryl or —C₅₋₁₀heterocyclyl, wherein the —C₆₋₁₀aryl, —C₅₋₁₀heteroaryl or —C₅₋₁₀heterocyclyl ring is optionally substituted by one, two or three groups independently selected from: —C₁₋₆alkyl (such as —CH₃), —OR⁵, —C₁₋₆alkoxy (such as —OCH₃), —C₁₋₆haloalkyl (such as —CF₃), —CN or halogen (such as chloro, bromo or fluoro),
   (iii) —C₅₋₁₀heteroaryl or —C₅₋₁₀heterocyclyl wherein the —C₅₋₁₀heteroaryl or —C₅₋₁₀heterocyclyl is optionally substituted by one, two or three groups independently selected from:
(a) —C₁₋₆alkyl (such as —CH₃), —C₁₋₆haloalkyl (such as —CF₃), —C₃₋₆cycloalkyl, —C₁₋₆alkoxy (such as —OCH₃), —OR⁵, —CN or halogen (such as chloro, bromo or fluoro),
(b) —C₆₋₁₀aryl (such as phenyl), —C₅₋₁₀heteroaryl or —C₅₋₁₀heterocyclyl wherein the —C₆₋₁₀aryl, —C₅₋₁₀heteroaryl or —C₅₋₁₀heterocyclyl ring is optionally substituted by one, two or three groups independently selected from: —C₁₋₆alkyl (such as —CH₃), —OR⁵, —C₁₋₆alkoxy (such as —OCH₃), —C₁₋₆haloalkyl (such as —CF₃), —CN or halogen (such as chloro, bromo or fluoro),
   Y represents —(CH₂)ₘ— or —O(CH₂)ₘ—,
   One of R² and R³ represents hydrogen and the other represents H, —C₁₋₆alkyl (such as —CH₃) or —C₃₋₆cycloalkyl,
   R⁴ represents H, —C₁₋₆alkyl, —C(═O)C₁₋₆alkyl, —C(═O)C₃₋₆cycloalkyl or —CO₂C₁₋₆alkyl,
   R⁵ represents —C₁₋₆haloalkyl (such as —CF₃) or —C₃₋₆cycloalkyl, and
   m represents 1-3,
   or a pharmaceutically acceptable salt thereof.

The said compounds have been found to inhibit SCD activity and may therefore be useful in the treatment of SCD-mediated diseases or conditions caused by or associated with an abnormal plasma lipid profile including dyslipidemia, hypoalphalipoproteinemia, hyperbetalipoproteinemia, hypercholesterolemia, hypertriglyceridemia, familial hypercholesterolemia, angina, ischemia, cardiac ischemia, stroke, myocardial infarction, atherosclerosis, obesity, Type I diabetes, Type II diabetes, insulin resistance, hyperinsulinaemia and metabolic syndrome; other cardiovascular diseases e.g. peripheral vascular disease, reperfusion injury, angioplastic restenosis, hypertension, vascular complications of diabetes, thrombosis, hepatic steatosis, non-alcoholic steatoheptatis (NASH) and other diseases related to accumulation of lipids in the liver; skin disorders e.g. eczema, acne, psoriasis, keloid scar formation or prevention, and diseases related to production or secretions from mucous membranes; cancer, neoplasia, malignancy, metastases, tumours (benign or malignant), carcinogenesis, hepatomas and the like; mild cognitive impairment (MCI), Alzheimer's Disease (AD), cerebral amyloid angiopathy (CAA) or dementia associated with Down Syndrome (DS) and other neurodegenerative diseases characterized by the formation or accumulation of amyloid plaques comprising Aβ42.

In one aspect, the invention provides a compound of formula (IA) which is a sub-set of compounds of the formula (I), for inhibiting SCD activity:

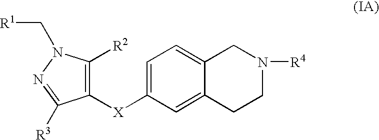

wherein:
X represents —CONH—, —NHCO— or —NHCONH—,
R¹ represents:
(i) a substituent selected from: H, —C₁₋₆alkyl or —C₃₋₆cycloalkyl,
(ii) —C₆₋₁₀aryl (such as phenyl) optionally substituted by one, two or three groups independently selected from:
(a) —C₁₋₆alkyl, —C₁₋₆haloalkyl (such as —CF₃), —C₃₋₆cycloalkyl, —C₁₋₆alkoxy, —OC₁₋₆haloalkyl (such as —OCF₃), —OR⁵, or halogen (such as chloro, bromo or fluoro),
(b) —C₆₋₁₀aryl (such as phenyl), —C₅₋₁₀heteroaryl or —C₅₋₁₀heterocyclyl, wherein the —C₆₋₁₀aryl, —C₅₋₁₀heteroaryl or —C₅₋₁₀heterocyclyl ring is optionally substituted by one, two or three groups independently selected from: —C₁₋₆alkyl, —OR⁵, —C₁₋₆alkoxy, —C₁₋₆haloalkyl (such as —CF₃) or halogen (such as chloro, bromo or fluoro),
   (iii) —C₅₋₁₀heteroaryl or —C₅₋₁₀heterocyclyl wherein the —C₅₋₁₀heteroaryl or —C₅₋₁₀heterocyclyl is optionally substituted by one, two or three groups independently selected from:
(a) —C₁₋₆alkyl, —C₁₋₆haloalkyl (such as —CF₃), —C₃₋₆cycloalkyl, —C₁₋₆alkoxy, —OC₁₋₆haloalkyl (such as —OCF₃), —OR⁵ or halogen (such as chloro, bromo or fluoro),
(b) —C₆₋₁₀aryl (such as phenyl), —C₅₋₁₀heteroaryl or —C₅₋₁₀heterocyclyl wherein the —C₆₋₁₀aryl, —C₅₋₁₀heteroaryl or —C₅₋₁₀heterocyclyl ring is optionally substituted by one, two or three groups independently selected from: —C₁₋₆alkyl, —OR⁵, —C₁₋₆alkoxy —C₁₋₆haloalkyl (such as —CF₃) or halogen (such as chloro, bromo or fluoro),
   One of R² and R³ represents hydrogen and the other represents H, —C₁₋₆alkyl (such as methyl) or —C₃₋₆cycloalkyl,
   R⁴ represents H, —C₁₋₆alkyl, —C(═O)C₁₋₆alkyl, —C(═O)C₃₋₆cycloalkyl or —CO₂C₁₋₆alkyl, and
   R⁵ represents, —C₁₋₆haloalkyl (such as —CF₃) or —C₃₋₆cycloalkyl,
   or a pharmaceutically acceptable salt thereof.

In one aspect of the invention, X represents —NHCO—. In another aspect of the invention, X represents —CONH—. In another aspect of the invention, X represents —NHCONH—.

In one aspect of the invention, R¹ represents —C₃₋₆cycloalkyl. In another aspect of the invention, R¹ represents cyclohexyl.

In another aspect of the invention, R¹ represents —C₆₋₁₀aryl optionally substituted by: one, two or three groups independently selected from:
(a) —C₁₋₆alkyl (such as —CH₃), —C₁₋₆haloalkyl (such as —CF₃), —C₃₋₆cycloalkyl, —C₁₋₆alkoxy (such as —OCH₃), —OR⁵, —CN or halogen (such as chloro, bromo or fluoro),
(b) —C₆₋₁₀aryl optionally substituted by one, two or three groups selected from: —C₁₋₆alkyl (such as —CH₃), —OR⁵, —C₁₋₆alkoxy (such as —OCH₃), —C₁₋₆haloalkyl (such as —CF₃), —CN or halogen (such as chloro, bromo or fluoro).

In another aspect of the invention, R¹ represents —C₆₋₁₀aryl optionally substituted by: one, two or three groups independently selected from:
(a) —C₁₋₆alkyl, —C₁₋₆haloalkyl (such as —CF₃), —C₃₋₆cycloalkyl, —C₁₋₆alkoxy, —OC₁₋₆haloalkyl (such as —OCF₃), —OR⁵ or halogen,
(b) —C₆₋₁₀aryl optionally substituted by one, two or three groups selected from: —C₁₋₆alkyl, —OR⁵, —C₁₋₆alkoxy, —C₁₋₆haloalkyl (such as —CF₃) or halogen.

In another aspect of the invention, R¹ represents phenyl optionally substituted by: one, two or three groups independently selected from:
(a) —C₁₋₆alkyl (such as —CH₃), —C₁₋₆haloalkyl (such as —CF₃), —C₃₋₆cycloalkyl, —C₁₋₆alkoxy (such as —OCH₃), —OR⁵, —CN or halogen (such as chloro, bromo or fluoro),
(b) phenyl optionally substituted by one, two or three groups selected from: —C₁₋₆alkyl (such as —CH₃), —OR⁵, —C₁₋₆alkoxy (such as —OCH₃), —C₁₋₆haloalkyl (such as —CF₃), —CN or halogen (such as chloro, bromo or fluoro).

In another aspect of the invention, R¹ represents phenyl optionally substituted by: one, two or three groups independently selected from:
(a) —C₁₋₆alkyl, —C₁₋₆haloalkyl (such as —CF₃), —C₃₋₆cycloalkyl, —C₁₋₆alkoxy, —OC₁₋₆haloalkyl (such as —OCF₃), —OR⁵ or halogen,
(b) phenyl optionally substituted by one, two or three groups selected from: —C₁₋₆alkyl, —OR⁵, —C₁₋₆alkoxy, —C₁₋₆haloalkyl (such as —CF₃) or halogen.

In another aspect of the invention, R¹ represents phenyl optionally substituted by one, two or three groups independently selected from:
(a) —C₁₋₆alkyl (such as —CH₃), —C₁₋₆haloalkyl (such as —CF₃), —C₁₋₆alkoxy (such as —OCH₃), —CN or halogen (such as chloro, bromo or fluoro),
(b) phenyl optionally substituted by one, two or three groups selected from: halogen (such as chloro, bromo or fluoro).

In another aspect of the invention, R¹ represents phenyl optionally substituted by one, two or three groups independently selected from:
(a) —C₁₋₆alkyl, —C₁₋₆haloalkyl (such as —CF₃), —C₁₋₆alkoxy or halogen,
(b) phenyl optionally substituted by one, two or three groups selected from: halogen.

In another aspect of the invention, R¹ represents phenyl optionally substituted by one, two or three groups independently selected from: —C₁₋₃alkyl (such as —CH₃), —C₁₋₃haloalkyl (such as —CF₃), —C₁₋₃alkoxy (such as —OCH₃), —CN or halogen (such as chloro, bromo or fluoro).

In another aspect of the invention, R¹ represents phenyl optionally substituted by one or two groups independently selected from: —C₁₋₃alkyl (such as —CH₃), —C₁₋₃haloalkyl (such as —CF₃), —C₁₋₃alkoxy (such as —OCH₃), —CN or halogen (such as chloro, bromo or fluoro).

In another aspect of the invention, R¹ represents phenyl optionally substituted by one, two or three groups independently selected from: —CH₃, —CF₃, —OCH₃, —CN or halogen (such as chloro, bromo or fluoro).

In another aspect of the invention, R¹ represents phenyl optionally substituted by one, two or three groups independently selected from: —CH₃, —CF₃, —OCH₃ or halogen.

In another aspect of the invention, R¹ represents phenyl optionally substituted by one or two groups independently selected from: —CH₃, —CF₃, —OCH₃, —CN or halogen (such as chloro or fluoro).

In another aspect of the invention, R¹ represents phenyl optionally substituted by one or two groups independently selected from: —CH₃, —CF₃, —OCH₃ or halogen (such as chloro or fluoro).

In another aspect of the invention, R¹ represents phenyl substituted by two chloro groups.

In another aspect of the invention, R¹ is phenyl substituted in the meta position, that is in the 3 position, and/or the para position, that is in the 4 position, by chloro i.e.

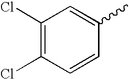

In another aspect of the invention, R¹ is phenyl substituted in the meta position, that is in the 3 position and 5 position, by halogen e.g. chloro i.e.

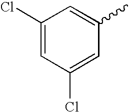

In another aspect of the invention, R¹ represents phenyl.

In another aspect of the invention, R¹ represents naphthyl optionally substituted by: one, two or three groups independently selected from:
(a) —C₁₋₆alkyl (such as —CH₃), —C₁₋₆haloalkyl (such as —CF₃), —C₃₋₆cycloalkyl, —C₁₋₆alkoxy (such as —OCH₃), —OR⁵, —CN or halogen (such as chloro, bromo or fluoro),
(b) phenyl optionally substituted by one, two or three groups selected from: —C₁₋₆alkyl (such as —CH₃), —OR⁵, —C₁₋₆alkoxy (such as —OCH₃), —C₁₋₆haloalkyl (such as —CF₃), —CN or halogen (such as chloro, bromo or fluoro).

In another aspect of the invention, R¹ represents naphthyl optionally substituted by: one, two or three groups independently selected from:
(a) —C₁₋₆alkyl, —C₁₋₆haloalkyl (such as —CF₃), —C₃₋₆cycloalkyl, —C₁₋₆alkoxy, —OC₁₋₆haloalkyl (such as —OCF₃), —OR⁵, halogen,
(b) phenyl optionally substituted by one, two or three groups selected from: —C₁₋₆alkyl, —OR⁵, —C₁₋₆alkoxy, —C₁₋₆haloalkyl (such as —CF₃) or halogen.

In another aspect of the invention, R¹ represents naphthyl.

In another aspect of the invention, R¹ represents —C₅₋₁₀heteroaryl or —C₅₋₁₀heterocyclyl wherein the —C₅₋₁₀heteroaryl or —C₅₋₁₀heterocyclyl is optionally substituted by one, two or three groups independently selected from:
(a) —C₁₋₆alkyl (such as —CH₃), —C₁₋₆haloalkyl (such as —CF₃), —C₃₋₆cycloalkyl, —C₁₋₆alkoxy (such as —OCH₃), —OR⁵, —CN or halogen (such as chloro, bromo or fluoro),
(b) —C₆₋₁₀aryl (such as phenyl), —C₅₋₁₀heteroaryl or —C₅₋₁₀heterocyclyl wherein the —C₆₋₁₀aryl, —C₅₋₁₀heteroaryl or —C₅₋₁₀heterocyclyl ring is optionally substituted by one, two or three groups independently selected from: —C₁₋₆alkyl (such as —CH₃), —OR⁵, —C₁₋₆alkoxy (such as —OCH₃), —C₁₋₆haloalkyl (such as —CF₃), —CN or halogen (such as chloro, bromo or fluoro).

In another aspect of the invention, R¹ represents —C₅₋₁₀heteroaryl or —C₅₋₁₀heterocyclyl wherein the —C₅₋₁₀heteroaryl or —C₅₋₁₀heterocyclyl is optionally substituted by one, two or three groups independently selected from:
(a) —C₁₋₆alkyl, C₁₋₆haloalkyl (such as —CF₃), —C₃₋₆cycloalkyl, —C₁₋₆alkoxy, —OC₁₋₆haloalkyl (such as —OCF₃), —OR⁵ or halogen (such as chloro, bromo or fluoro),
(b) —C₆₋₁₀aryl (such as phenyl), —C₅₋₁₀heteroaryl or —C₅₋₁₀heterocyclyl wherein the —C₆₋₁₀aryl, —C₅₋₁₀heteroaryl or —C₅₋₁₀heterocyclyl ring is optionally substituted by one, two or three groups independently selected from: —C₁₋₆alkyl, —OR⁵, —C₁₋₆alkoxy, —C₁₋₆haloalkyl (such as —CF₃) or halogen (such as chloro, bromo or fluoro).

In another aspect of the invention, R¹ represents —C₅₋₁₀heteroaryl or —C₅₋₁₀heterocyclyl wherein the —C₅₋₁₀heteroaryl or —C₅₋₁₀heterocyclyl is optionally substituted by one, two or three groups independently selected from:
(a) —C₁₋₆alkyl (such as —CH₃), —C₁₋₆haloalkyl (such as —CF₃), —C₃₋₆cycloalkyl, —C₁₋₆alkoxy (such as —OCH₃), —OR⁵, —CN or halogen (such as chloro, bromo or fluoro),
(b) phenyl optionally substituted by one, two or three groups selected from: —C₁₋₆alkyl (such as —CH₃), —OR⁵, —C₁₋₆alkoxy (such as —OCH₃), —C₁₋₆haloalkyl (such as —CF₃), —CN or halogen (such as chloro, bromo or fluoro).

In one aspect of the invention, Y represents —(CH₂)ₘ—. In another aspect of the invention, Y represents —O(CH₂)ₘ₋₁. In another aspect of the invention, Y represents —CH₂—. In another aspect of the invention, Y represents —OCH₂—.

In one aspect of the invention, R² represents hydrogen or —C₁₋₆alkyl. In another aspect of the invention, R² represents hydrogen or —C₁₋₃alkyl. In another aspect of the invention, R² represents hydrogen or methyl (—CH₃). In another aspect of the invention, R² represents hydrogen. In another aspect of the invention, R² represents methyl (—CH₃). In another aspect of the invention, R² represents —C₃₋₆cycloalkyl.

In one aspect of the invention, R³ represents hydrogen or —C₁₋₆alkyl. In another aspect of the invention, R³ represents hydrogen or —C₁₋₃alkyl. In another aspect of the invention, R³ represents hydrogen or methyl (—CH₃). In another aspect of the invention, R³ represents hydrogen. In another aspect of the invention, R³ represents methyl (—CH₃). In another aspect of the invention, R³ represents —C₃₋₆cycloalkyl

In one aspect of the invention, R² represents —C₁₋₃alkyl and R³ represents hydrogen.

In another aspect of the invention, R² represents methyl (—CH₃) and R³ represents hydrogen.

In one aspect of the invention, R³ represents —C₁₋₃alkyl and R² represents hydrogen.

In another aspect of the invention, R³ represents methyl (—CH₃) and R² represents hydrogen.

In one aspect of the invention, R⁴ represents hydrogen or —C₁₋₆alkyl. In another aspect of the invention, R⁴ represents hydrogen or —C₁₋₃alkyl. In one aspect of the invention, R⁴ represents hydrogen or —C₃H₇ (propyl). In another aspect of the invention, R⁴ represents hydrogen. In another aspect of the invention, R⁴ represents —C₃H₇ (propyl).

In another aspect of the invention, R⁴ represents —C(═O)C₁₋₆alkyl. In another aspect of the invention, R⁴ represents —C(═O)CH₃. In another aspect of the invention, R⁴ represents —C(═O)C₃H₇. In another aspect of the invention, R⁴ represents —C(═O)C₂H₄—CH(CH₃)₂.

In another aspect of the invention, R⁴ represents —C(═O)C₃₋₆cycloalkyl. In another aspect of the invention, R⁴ represents —C(═O)C₅cycloalkyl. In another aspect of the invention, R⁴ represents —C(═O)cyclopentyl.

In another aspect of the invention, R⁴ represents —CO₂C₁₋₆alkyl. In another aspect of the invention, R⁴ represents —CO₂C(CH₃)₃.

In one aspect of the invention, R⁵ represents —C₁₋₆haloalkyl. In another aspect of the invention, R⁵ represents —C₃₋₆cycloalkyl.

In one aspect of the invention, m represents 1. In another aspect of the invention, m represents 2.

In one aspect of the invention, when:

R¹ represents phenyl substituted in the 2-position by (CH₃)₂CHCH₂O— then said phenyl is not substituted by chloro in the 5-position.

Each of the aspects of the invention are independent unless stated otherwise. Nevertheless the skilled person will understand that all the permutations of the aspects of described are within the scope of the invention. Thus it is to be understood that the present invention covers all combinations of suitable, convenient and exemplified groups described herein. For example, in one aspect the invention provides a compound of formula (I) wherein X represents —NHCO— and R⁴ represents H.

Certain compounds of formula (I) may exist in stereoisomeric forms (e.g. they may contain one or more asymmetric carbon atoms). The individual stereoisomers (enantiomers and diastereomers) and mixtures of these are included within the scope of the present invention. The invention also extends to conformational isomers of compounds of formula (I) and any geometric (cis and/or trans) isomers of said compounds. Likewise, it is understood that compounds of formula (I) may exist in tautomeric forms other than that shown in the formula and these are also included within the scope of the present invention.

It will be appreciated that racemic compounds of formula (I) may be optionally resolved into their individual enantiomers. Such resolutions may conveniently be accomplished by standard methods known in the art. For example, a racemic compound of formula (I) may be resolved by chiral preparative HPLC.

It will also be appreciated that compounds of the invention which exist as polymorphs, and mixtures thereof, are within the scope of the present invention.

As used herein, the term “alkyl” refers to straight or branched hydrocarbon chains containing the specified number of carbon atoms. For example, C₁₋₆alkyl means a straight or branched alkyl containing at least 1, and at most 6, carbon atoms. Examples of “alkyl” as used herein include, but are not limited to, methyl, ethyl, n-propyl, n-butyl, n-pentyl, n-hexyl, isobutyl, isopropyl, t-butyl and 1,1-dimethylpropyl. However, when a moiety is defined such that alkyl bears a substituent it will be clear to the skilled person from the context that alkyl may include alkylene, for example methylene (—CH₂—), ethylene (—CH₂CH₂—) and propylene (—CH₂CH₂CH₂—).

As used herein, the term “alkoxy” refers to a straight or branched alkoxy group containing the specified number of carbon atoms. For example, C₁₋₆alkoxy means a straight or branched alkoxy group containing at least 1, and at most 6, carbon atoms. Examples of “alkoxy” as used herein include, but are not limited to, methoxy, ethoxy, propoxy, prop-2-oxy, butoxy, but-2-oxy, 2-methylprop-1-oxy, 2-methylprop-2-oxy, pentoxy and hexyloxy. The point of attachment may be on the oxygen or carbon atom.

As used herein, the term “halogen” or “halo” refers to a fluorine (fluoro), chlorine (chloro), bromine (bromo) or iodine (iodo) atom.

As used herein, the term “haloalkyl” refers to an alkyl group having one or more carbon atoms and wherein at least one hydrogen atom is replaced with a halogen atom, for example a trifluoromethyl group and the like.

As used herein, the term “cycloalkyl” refers to a saturated cyclic group containing 3 to 10 carbon ring-atoms, such as 3 to 6 carbon ring-atoms. Examples include cyclopropyl, cyclopentyl and cyclohexyl.

As used herein, the term “—C₅₋₁₀heteroaryl” refers to an aromatic cyclic group containing 5 to 10 ring-atoms 1, 2, 3 or 4 of which are hetero-atoms independently selected from nitrogen, oxygen and sulphur and the remaining ring-atoms are carbon, e.g. benzothiophene. This definition includes both monocyclic and bicyclic ring systems and bicyclic structures at least a portion of which is aromatic and the other part is saturated, partially or fully unsaturated.

As used herein, the term ‘aryl’ means an aromatic carbocyclic moiety. The definition includes both monocyclic and bicyclic ring systems and bicyclic structures at least a portion of which is aromatic and the other part is saturated, partially or fully unsaturated. Examples of aromatic, aryl groups include naphthyl, anthryl, phenanthryl, indanyl, indenyl, azulenyl, azulanyl, fluorenyl, phenyl and naphthyl, and more specifically phenyl.

As used herein, the term “—C₅₋₁₀heterocyclyl” refers to a cyclic group containing 5 to 10 ring-atoms 1, 2, 3 or 4 of which are hetero-atoms independently selected from nitrogen, oxygen and sulphur and the remaining ring-atoms are carbon, wherein said cyclic group is saturated, partially or fully unsaturated but, which is not aromatic. This definition includes bicyclic structures provided the moiety is non-aromatic.

Examples of heterocyclyl and heteroaromatic groups include: furyl, thienyl, pyrrolyl, pyrrolinyl, pyrrolidinyl, imidazolyl, dioxolanyl, oxazolyl, thiazolyl, imidazolyl, imidazolinyl, imidazolidinyl, pyrazolyl, pyrazolinyl, pyrazolidinyl, isoxazolyl, isothiazolyl, oxadiazolyl, triazolyl, thiadiazolyl, pyranyl, pyridyl, piperidinyl, homopiperazinyl, dioxanyl, morpholino, dithianyl, thiomorpholino, pyridazinyl, pyrimidinyl, pyrazinyl, piperazinyl, sulfolanyl, tetrazolyl, triazinyl, azepinyl, oxazepinyl, thiazepinyl, diazepinyl and thiazolinyl, benzimidazolyl, benzoxazolyl, imidazopyridinyl, benzoxazinyl, benzothiazinyl, benzothiophenyl oxazolopyridinyl, benzofuranyl, quinolinyl, quinazolinyl, quinoxalinyl, dihydroquinazolinyl, benzothiazolyl, phthalimido, benzofuranyl, benzodiazepinyl, indolyl and isoindolyl.

As used herein, the term “substituted” refers to substitution with the named substituent or substituents, multiple degrees of substitution being allowed unless otherwise stated.

For the avoidance of doubt, the term “independently” means that where more than one substituent is selected from a number of possible substituents, those substituents may be the same or different.

As used herein, the term “pharmaceutically acceptable” means a compound which is suitable for pharmaceutical use.

Salts of compounds of formula (I) which are suitable for use in medicine are those wherein the counterion is pharmaceutically acceptable. However, salts having non-pharmaceutically acceptable counterions are within the scope of the present invention, for example, for use as intermediates in the preparation of other compounds of formula (I) and their pharmaceutically acceptable salts.

Suitable pharmaceutically acceptable salts will be apparent to those skilled in the art and include for example acid addition salts formed with inorganic acids e.g. hydrochloric, hydrobromic, sulfuric, nitric or phosphoric acid; and organic acids e.g. succinic, maleic, malic, mandelic, acetic, fumaric, glutamic, lactic, citric, tartaric, benzoic, benzenesulfonic, p-toluenesulfonic, methanesulfonic, ethanesulfonic or naphthalenesulfonic acid. Other non-pharmaceutically acceptable salts e.g. oxalates, may be used, for example in the isolation of compounds of formula (I) and are included within the scope of this invention. Reference is made to Berge et al. J. Pharm. Sci., 1977, 66, 1-19.

Certain of the compounds of formula (I) may form acid addition salts with one or more equivalents of the acid. The present invention includes within its scope all possible stoichiometric and non-stoichiometric forms thereof.

Solvates of the compounds of formula (I) and solvates of the salts of the compounds of formula (I) are included within the scope of the present invention.

As used herein, the term “solvate” refers to a complex of variable stoichiometry formed by a solute (in this invention, a compound of formula (I) or a salt thereof) and a solvent. Such solvents for the purpose of the invention may not interfere with the biological activity of the solute. Examples of suitable solvents include, but are not limited to, water, methanol, ethanol and acetic acid. Preferably the solvent used is a pharmaceutically acceptable solvent. Most preferably the solvent used is water and the solvate may also be referred to as a hydrate.

Solvates of compounds of formula (I) which are suitable for use in medicine are those wherein the solvent is pharmaceutically acceptable. However, solvates having non-pharmaceutically acceptable solvents are within the scope of the present invention, for example, for use as intermediates in the preparation of other compounds of formula (I) and their pharmaceutically acceptable salts.

Prodrugs of the compounds of formula (I) are included within the scope of the present invention.

As used herein, the term “prodrug” means a compound which is converted within the body, e.g. by hydrolysis in the blood, into its active form that has medical effects. Pharmaceutically acceptable prodrugs are described in T. Higuchi and V. Stella, Prodrugs as Novel Delivery Systems, Vol. 14 of the A.C.S. Symposium Series, and in Edward B. Roche, ed., Bioreversible Carriers in Drug Design, American Pharmaceutical Association and Pergamon Press, 1987 and in D. Fleishner, S. Ramon and H. Barba “Improved oral drug delivery: solubility limitations overcome by the use of prodrugs”, Advanced Drug Delivery Reviews (1996) 19(2) 115-130. Prodrugs are any covalently bonded carriers that release a compound of structure (I) in vivo when such prodrug is administered to a patient. Prodrugs are generally prepared by modifying functional groups in a way such that the modification is cleaved in vivo yielding the parent compound. Prodrugs may include, for example, compounds of this invention wherein hydroxy or amine groups are bonded to any group that, when administered to a patient, cleaves to form the hydroxy or amine groups. Thus, representative examples of prodrugs include (but are not limited to) phosphonate, carbamate, acetate, formate and benzoate derivatives of hydroxy and amine functional groups of the compounds of formula (I).

Phosphonates and carbamates may be active in their own right and/or be hydrolysable under in vivo conditions in the human body. Suitable pharmaceutically acceptable in vivo hydrolysable ester groups include those which break down readily in the human body to leave the parent acid or its salt. A phosphonate is formed by reaction with phosphorous (phosphonic) acid, by methods well known in the art. For example, phosphonates may be derivatives such as RP(O)(OR)₂ and the like. A carbamate is an ester of carbamic acid.

In one aspect of the invention there is provided a compound, or a pharmaceutically acceptable salt thereof, wherein the compound is selected from the group consisting of:
N-{1-[(3,4-Dichlorophenyl)methyl]-1H-pyrazol-4-yl}-1,2,3,4-tetrahydro-6-isoquinolinecarboxamide,
N-[1-(1-naphthalenylmethyl)-1H-pyrazol-4-yl]-1,2,3,4-tetrahydro-6-isoquinolinecarboxamide,
N-{1-[(2,4-dichlorophenyl)methyl]-1H-pyrazol-4-yl}-1,2,3,4-tetrahydro-6-isoquinolinecarboxamide,
N-{1-[(2-methylphenyl)methyl]-1H-pyrazol-4-yl}-1,2,3,4-tetrahydro-6-isoquinolinecarboxamide,
N-[1-(cyclohexylmethyl)-1H-pyrazol-4-yl]-1,2,3,4-tetrahydro-6-isoquinolinecarboxamide,
N-(1-{[4-(trifluoromethyl)phenyl]methyl}-1H-pyrazol-4-yl)-1,2,3,4-tetrahydro-6-isoquinolinecarboxamide,
N-{1-[(3-chlorophenyl)methyl]-1H-pyrazol-4-yl}-1,2,3,4-tetrahydro-6-isoquinolinecarboxamide,
N-{1-[(2-chlorophenyl)methyl]-1H-pyrazol-4-yl}-1,2,3,4-tetrahydro-6-isoquinolinecarboxamide,
N-(1-{[3-(methyloxy)phenyl]methyl}-1H-pyrazol-4-yl)-1,2,3,4-tetrahydro-6-isoquinolinecarboxamide,
N-{1-[(3-fluorophenyl)methyl]-1H-pyrazol-4-yl}-1,2,3,4-tetrahydro-6-isoquinolinecarboxamide,
N-(1-{[3-(trifluoromethyl)phenyl]methyl}-1H-pyrazol-4-yl)-1,2,3,4-tetrahydro-6-isoquinolinecarboxamide,
N-{1-[(4-chlorophenyl)methyl]-1H-pyrazol-4-yl}-1,2,3,4-tetrahydro-6-isoquinolinecarboxamide,
N-{1-[(3-methylphenyl)methyl]-1H-pyrazol-4-yl}-1,2,3,4-tetrahydro-6-isoquinolinecarboxamide,
N-{1-[(4-methylphenyl)methyl]-1H-pyrazol-4-yl}-1,2,3,4-tetrahydro-6-isoquinolinecarboxamide,
N-{1-[(4-fluorophenyl)methyl]-1H-pyrazol-4-yl}-1,2,3,4-tetrahydro-6-isoquinolinecarboxamide,
1-[(3,4-Dichlorophenyl)methyl]-N-(1,2,3,4-tetrahydro-6-isoquinolinyl)-1H-pyrazole-4-carboxamide hydrochloride,
1-(phenylmethyl)-N-(1,2,3,4-tetrahydro-6-isoquinolinyl)-1H-pyrazole-4-carboxamide hydrochloride,
1-[(3,4-dichlorophenyl)methyl]-5-methyl-N-(1,2,3,4-tetrahydro-6-isoquinolinyl)-1H-pyrazole-4-carboxamide,
1-[(3,4-dichlorophenyl)methyl]-3-methyl-N-(1,2,3,4-tetrahydro-6-isoquinolinyl)-1H-pyrazole-4-carboxamide hydrochloride,
N-(1,2,3,4-tetrahydro-6-isoquinolinyl)-1-{[3-(trifluoromethyl)phenyl]methyl}-1H-pyrazole-4-carboxamide,
1-(1-naphthalenylmethyl)-N-(1,2,3,4-tetrahydro-6-isoquinolinyl)-1H-pyrazole-4-carboxamide,
1-[(4-fluorophenyl)methyl]-N-(1,2,3,4-tetrahydro-6-isoquinolinyl)-1H-pyrazole-4-carboxamide,
N-{1-[(3,4-dichlorophenyl)methyl]-1H-pyrazol-4-yl}-2-propyl-1,2,3,4-tetrahydro-6-isoquinolinecarboxamide,
1,1-Dimethylethyl 6-[({1-[(3,4-dichlorophenyl)methyl]-1H-pyrazol-4-yl}amino)carbonyl]-3,4-dihydro-2(1H)-isoquinolinecarboxylate,
1,1-dimethylethyl 6-[({1-[(3-cyanophenyl)methyl]-1H-pyrazol-4-yl}amino)carbonyl]-3,4-dihydro-2(1H)-isoquinolinecarboxylate,
2-acetyl-N-{1-[(3,4-dichlorophenyl)methyl]-1H-pyrazol-4-yl}-1,2,3,4-tetrahydro-6-isoquinolinecarboxamide,
2-butanoyl-N-{1-[(3,4-dichlorophenyl)methyl]-1H-pyrazol-4-yl}-1,2,3,4-tetrahydro-6-isoquinolinecarboxamide,
2-(cyclopentylcarbonyl)-N-{1-[(3,4-dichlorophenyl)methyl]-1H-pyrazol-4-yl}-1,2,3,4-tetrahydro-6-isoquinolinecarboxamide,
N-{1-[(3,4-dichlorophenyl)methyl]-1H-pyrazol-4-yl}-2-(4-methylpentanoyl)-1,2,3,4-tetrahydro-6-isoquinolinecarboxamide,
N-(1-{[(3,4-dichlorophenyl)oxy]methyl}-1H-pyrazol-4-yl)-1,2,3,4-tetrahydro-6-isoquinolinecarboxamide hydrochloride,
N-[1-({[3-(trifluoromethyl)phenyl]oxy}methyl)-1H-pyrazol-4-yl]-1,2,3,4-tetrahydro-6-isoquinolinecarboxamide hydrochloride,
N-(1-{[(2,5-dichlorophenyl)oxy]methyl}-1H-pyrazol-4-yl)-1,2,3,4-tetrahydro-6-isoquinolinecarboxamide hydrochloride,
N-(1-{[(2,4-dichlorophenyl)oxy]methyl}-1H-pyrazol-4-yl)-1,2,3,4-tetrahydro-6-isoquinolinecarboxamide hydrochloride, or
N-(1-{[(3-chlorophenyl)oxy]methyl}-1H-pyrazol-4-yl)-1,2,3,4-tetrahydro-6-isoquinolinecarboxamide hydrochloride.

In another aspect of the invention there is provided a compound, selected from the group consisting of:
N-{1-[(3,4-Dichlorophenyl)methyl]-1H-pyrazol-4-yl}-1,2,3,4-tetrahydro-6-isoquinolinecarboxamide hydrochloride,
N-[1-(1-naphthalenylmethyl)-1H-pyrazol-4-yl]-1,2,3,4-tetrahydro-6-isoquinolinecarboxamide hydrochloride,
N-{1-[(2,4-dichlorophenyl)methyl]-1H-pyrazol-4-yl}-1,2,3,4-tetrahydro-6-isoquinolinecarboxamide,
N-{1-[(2-methylphenyl)methyl]-1H-pyrazol-4-yl}-1,2,3,4-tetrahydro-6-isoquinolinecarboxamide,
N-[1-(cyclohexylmethyl)-1H-pyrazol-4-yl]-1,2,3,4-tetrahydro-6-isoquinolinecarboxamide,
N-(1-{[4-(trifluoromethyl)phenyl]methyl}-1H-pyrazol-4-yl)-1,2,3,4-tetrahydro-6-isoquinolinecarboxamide,
N-{1-[(3-chlorophenyl)methyl]-1H-pyrazol-4-yl}-1,2,3,4-tetrahydro-6-isoquinolinecarboxamide,
N-{1-[(2-chlorophenyl)methyl]-1H-pyrazol-4-yl}-1,2,3,4-tetrahydro-6-isoquinolinecarboxamide,
N-(1-{[3-(methyloxy)phenyl]methyl}-1H-pyrazol-4-yl)-1,2,3,4-tetrahydro-6-isoquinolinecarboxamide,
N-{1-[(3-fluorophenyl)methyl]-1H-pyrazol-4-yl}-1,2,3,4-tetrahydro-6-isoquinolinecarboxamide,
N-(1-{[3-(trifluoromethyl)phenyl]methyl}-1H-pyrazol-4-yl)-1,2,3,4-tetrahydro-6-isoquinolinecarboxamide,
N-{1-[(4-chlorophenyl)methyl]-1H-pyrazol-4-yl}-1,2,3,4-tetrahydro-6-isoquinolinecarboxamide,
N-{1-[(3-methylphenyl)methyl]-1H-pyrazol-4-yl}-1,2,3,4-tetrahydro-6-isoquinolinecarboxamide,
N-{1-[(4-methylphenyl)methyl]-1H-pyrazol-4-yl}-1,2,3,4-tetrahydro-6-isoquinolinecarboxamide,
N-{1-[(4-fluorophenyl)methyl]-1H-pyrazol-4-yl}-1,2,3,4-tetrahydro-6-isoquinolinecarboxamide,
1-[(3,4-Dichlorophenyl)methyl]-N-(1,2,3,4-tetrahydro-6-isoquinolinyl)-1H-pyrazole-4-carboxamide hydrochloride,
1-(phenylmethyl)-N-(1,2,3,4-tetrahydro-6-isoquinolinyl)-1H-pyrazole-4-carboxamide hydrochloride,
1-[(3,4-dichlorophenyl)methyl]-5-methyl-N-(1,2,3,4-tetrahydro-6-isoquinolinyl)-1H-pyrazole-4-carboxamide hydrochloride,
1-[(3,4-dichlorophenyl)methyl]-3-methyl-N-(1,2,3,4-tetrahydro-6-isoquinolinyl)-1H-pyrazole-4-carboxamide hydrochloride,
N-(1,2,3,4-tetrahydro-6-isoquinolinyl)-1-{[3-(trifluoromethyl)phenyl]methyl}-1H-pyrazole-4-carboxamide,
1-(1-naphthalenylmethyl)-N-(1,2,3,4-tetrahydro-6-isoquinolinyl)-1H-pyrazole-4-carboxamide,
1-[(4-fluorophenyl)methyl]-N-(1,2,3,4-tetrahydro-6-isoquinolinyl)-1H-pyrazole-4-carboxamide,
N-{1-[(3,4-dichlorophenyl)methyl]-1H-pyrazol-4-yl}-2-propyl-1,2,3,4-tetrahydro-6-isoquinolinecarboxamide hydrochloride,
1,1-Dimethylethyl 6-[({1-[(3,4-dichlorophenyl)methyl]-1H-pyrazol-4-yl}amino)carbonyl]-3,4-dihydro-2(1H)-isoquinolinecarboxylate,
1,1-dimethylethyl 6-[({1-[(3-cyanophenyl)methyl]-1H-pyrazol-4-yl}amino)carbonyl]-3,4-dihydro-2(1H)-isoquinolinecarboxylate,
2-acetyl-N-{1-[(3,4-dichlorophenyl)methyl]-1H-pyrazol-4-yl}-1,2,3,4-tetrahydro-6-isoquinolinecarboxamide,
2-butanoyl-N-{1-[(3,4-dichlorophenyl)methyl]-1H-pyrazol-4-yl}-1,2,3,4-tetrahydro-6-isoquinolinecarboxamide,
2-(cyclopentylcarbonyl)-N-{1-[(3,4-dichlorophenyl)methyl]-1H-pyrazol-4-yl}-1,2,3,4-tetrahydro-6-isoquinolinecarboxamide,
N-{1-[(3,4-dichlorophenyl)methyl]-1H-pyrazol-4-yl}-2-(4-methylpentanoyl)-1,2,3,4-tetrahydro-6-isoquinolinecarboxamide,
N-(1-{[(3,4-dichlorophenyl)oxy]methyl}-1H-pyrazol-4-yl)-1,2,3,4-tetrahydro-6-isoquinolinecarboxamide hydrochloride,
N-[1-({[3-(trifluoromethyl)phenyl]oxy}methyl)-1H-pyrazol-4-yl]-1,2,3,4-tetrahydro-6-isoquinolinecarboxamide hydrochloride,
N-(1-{[(2,5-dichlorophenyl)oxy]methyl}-1H-pyrazol-4-yl)-1,2,3,4-tetrahydro-6-isoquinolinecarboxamide hydrochloride,
N-(1-{[(2,4-dichlorophenyl)oxy]methyl}-1H-pyrazol-4-yl)-1,2,3,4-tetrahydro-6-isoquinolinecarboxamide hydrochloride, or
N-(1-{[(3-chlorophenyl)oxy]methyl}-1H-pyrazol-4-yl)-1,2,3,4-tetrahydro-6-isoquinolinecarboxamide hydrochloride.

The compounds of the invention have been found to inhibit SCD activity and may therefore be useful in regulating lipid levels, e.g. plasma lipid levels. Diseases or conditions caused by or associated with an abnormal plasma lipid profile and for the treatment of which the compounds of the invention may be useful include; dyslipidemia hypoalphalipoproteinemia, hyperbetalipoproteinemia, hypercholesterolemia, hypertriglyceridemia, familial hypercholesterolemia, angina, ischemia, cardiac ischemia, stroke, myocardial infarction, atherosclerosis, obesity, Type I diabetes, Type II diabetes, insulin resistance, hyperinsulinaemia and metabolic syndrome. Other cardiovascular diseases for which the compounds of the present invention are useful include peripheral vascular disease, reperfusion injury, angioplastic restenosis, hypertension, vascular complications of diabetes and thrombosis. Other diseases or conditions include hepatic steatosis, non-alcoholic steatohepatitis (NASH) and other diseases related to accumulation of lipids in the liver.

The compounds of the invention may also be useful in the treatment of skin disorders e.g. eczema, acne, psoriasis, keloid scar formation or prevention, and diseases related to production or secretions from mucous membranes.

The compounds of the invention may also be useful in the treatment of cancer, neoplasia, malignancy, metastases, tumours (benign or malignant), carcinogenesis, hepatomas and the like.

The compounds of the invention may also be useful in the treatment of mild cognitive impairment (MCI), Alzheimer's disease (AD), cerebral amyloid angiopathy (CAA) or dementia associated with Down Syndrome (DS) and other neurodegenerative diseases characterized by the formation or accumulation of amyloid plaques comprising Aβ42.

Within the context of the present invention, the terms describing the indications used herein are classified in the Merck Manual of Diagnosis and Therapy, 17^{th} Edition and/or the International Classification of Diseases 10^{th} Edition (ICD-10). The various subtypes of the disorders mentioned herein are contemplated as part of the present invention.

In one aspect, the invention provides a compound of formula (I) or a pharmaceutically acceptable salt thereof for use in medical therapy.

In one aspect, the invention provides the use of a compound of formula (I) or a pharmaceutically acceptable salt thereof for the manufacture of a medicament for treating and/or preventing a disease or a condition susceptible to amelioration by an SCD inhibitor.

In another aspect, the invention provides the use of a compound of formula (I) or a pharmaceutically acceptable salt thereof for the manufacture of a medicament for treating and/or preventing acne, cancer, dyslipidemia, hypertriglyceridemia, atherosclerosis, obesity, Type II diabetes, insulin resistance, hyperinsulinaemia, hepatic steatosis and/or non-alcoholic steatohepatitis (NASH).

In another aspect, the invention provides the use of a compound of formula (I) or a pharmaceutically acceptable salt thereof for the manufacture of a medicament for treating and/or preventing acne, cancer, dyslipidemia, atherosclerosis, insulin resistance, hyperinsulinaemia, Type II diabetes and/or hepatic steatosis.

In another aspect, the invention provides the use of a compound of formula (I) or a pharmaceutically acceptable salt thereof for the manufacture of a medicament for treating and/or preventing acne.

In one aspect, the invention provides a compound of formula (I) or a pharmaceutically acceptable salt thereof for use in treating and/or preventing a disease or a condition susceptible to amelioration by an SCD inhibitor in a mammal, including human.

In another aspect, the invention provides a compound of formula (I) or a pharmaceutically acceptable salt thereof for use in treating and/or preventing acne, cancer, dyslipidemia, hypertriglyceridemia, atherosclerosis, obesity, Type II diabetes, insulin resistance, hyperinsulinaemia, hepatic steatosis and/or non-alcoholic steatohepatitis (NASH).

In another aspect, the invention provides a compound of formula (I) or a pharmaceutically acceptable salt thereof for use in treating and/or preventing acne, cancer, dyslipidemia, atherosclerosis, insulin resistance, hyperinsulinaemia, Type II diabetes and/or hepatic steatosis.

In another aspect, the invention provides a compound of formula (I) or a pharmaceutically acceptable salt thereof for use in treating and/or preventing acne.

In one aspect, the invention provides a method for treating and/or preventing a disease or a condition susceptible to amelioration by an SCD inhibitor, which method comprises administering to a subject, for example a mammal, including human, a therapeutically effective amount of a compound of formula (I) or a pharmaceutically acceptable salt thereof.

In another aspect, the invention provides a method for treating and/or preventing a acne, cancer, dyslipidemia, hypertriglyceridemia, atherosclerosis, obesity, Type II diabetes, insulin resistance, hyperinsulinaemia, hepatic steatosis and/or non-alcoholic steatohepatitis (NASH), which method comprises administering to a subject, for example a mammal, including human, a therapeutically effective amount of a compound of formula (I) or a pharmaceutically acceptable salt thereof.

In another aspect, the invention provides a method for treating and/or preventing acne, cancer, dyslipidemia, atherosclerosis, insulin resistance, hyperinsulinaemia, Type II diabetes and/or hepatic steatosis, which method comprises administering to a subject, for example a mammal, including human, a therapeutically effective amount of a compound of formula (I) or a pharmaceutically acceptable salt thereof.

In another aspect, the invention provides a method for treating and/or preventing acne, which method comprises administering to a subject, for example a mammal, including human, a therapeutically effective amount of a compound of formula (I) or a pharmaceutically acceptable salt thereof.

It will be appreciated that reference to “treatment” and “therapy” includes acute treatment or prophylaxis as well as the alleviation of established symptoms.

Since the compounds of the invention are intended for use in pharmaceutical compositions it will readily be understood that they are each preferably provided in substantially pure form, for example at least 60% pure, more suitably at least 75% pure and preferably at least 85%, especially at least 98% pure (% are on a weight for weight basis). Impure preparations of the compounds may be used for preparing the more pure forms used in the pharmaceutical compositions; these less pure preparations of the compounds should contain at least 1%, more suitably at least 5% and preferably from 10 to 59% of a compound of the invention.

Processes for the preparation of the compounds of formula (I) form further aspects of the invention. R¹, R², R³ and R⁴ are as defined above unless otherwise specified. Throughout the specification, general formulae are designated by Roman numerals (I), (II), (III), (IV) etc.

In certain instances final compounds of formula (I) can be converted into other compounds of formula (I) by techniques known to those in the art, for example, carboxylic acid substituents can be converted to esters or amides by routine techniques.

In a general process, compounds of formula (I), wherein X represents —NHCO— and R⁴ represents H (formula (Ia)), may be prepared according to reaction scheme 1 by reacting compounds of formula (III) and compounds of formula (IV), wherein P¹ represents a suitable nitrogen protecting group such as Boc, to form a compound of formula (II). The reaction is suitably carried out in the presence of a coupling reagent such as HATU, EDCl and/or HOBt, in a suitable solvent such as DCM (suitably at room temperature to reflux) or DMF (suitably at room temperature to 80° C.), and is followed by deprotection of compounds of formula (II) under acidic conditions such as hydrochloric acid or trifluoracetic acid.

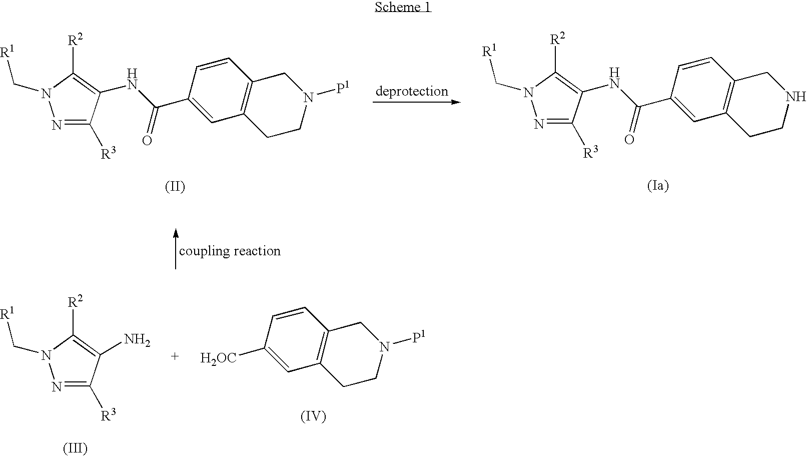

Accordingly, in one aspect the invention provides a process for the preparation of compounds of the formula (Ia) by reacting compounds of formula (III), wherein R¹, R² and R² are defined above, with compounds of formula (IV), wherein P¹ is defined above, in the presence of a coupling agent, followed by deprotection of compounds of formula (II).

Compounds of formula (II) may be also prepared according to reaction scheme 2 by reacting compounds of formula (VI) and compounds of formula (IV), wherein P¹ represents a suitable nitrogen protecting group such as Boc, to form a compound of formula (V). The reaction is suitably carried out in the presence of a coupling reagent such EDCl and HOBt, in a suitable solvent such as DMF (suitably at room temperature) and is followed by alkylation of compounds of formula (V) with an alkylating agent in a suitable solvent such as DMF (suitably at room temperature) in the presence of a base such as potassium carbonate.

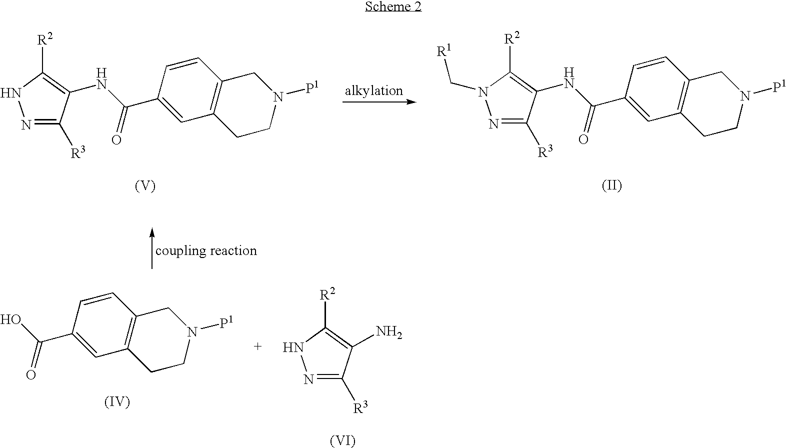

Compounds of formula (I), wherein X represents —NHCO— and R⁴ represents —C₁₋₆alkyl (formula (Ib)), may be prepared according to reaction scheme 3 by reacting compounds of formula (III) and compounds of formula (IVa), in the presence of a coupling reagent such as HATU, EDCl and/or HOBt, in a suitable solvent such as DCM (suitably at room temperature to reflux).

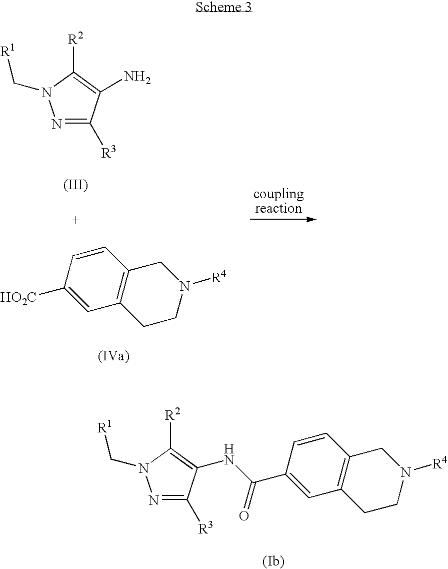

Accordingly, in one aspect the invention provides a process for the preparation of compounds of the formula (Ib) by reacting compounds of formula (III), wherein R¹, R² and R³ are defined above, with compounds of formula (IVa), wherein R⁴ is defined above, in the presence of a coupling agent.

Compounds of formula (I), wherein X represents —CONH— and R⁴ represents hydrogen (formula (Ic)), may be prepared according to reaction scheme 4 by reacting compounds of formula (VIII) and compounds of formula (IX), wherein P¹ represents a suitable nitrogen protecting group such as Boc, to form a compound of formula (VII). The reaction is suitably carried out in the presence of a coupling reagent such as HATU, EDCl and/or HOBt, in a suitable solvent such as DCM (suitably at room temperature to reflux) or DMF (suitably at room temperature to 80° C.), and is followed by deprotection of compounds of formula (VII) under acidic conditions such as hydrochloric acid or trifluoracetic acid.

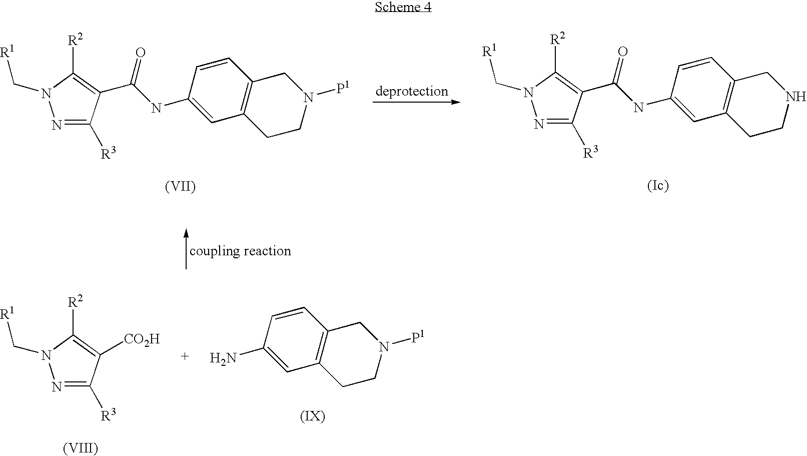

Accordingly, in one aspect the invention provides a process for the preparation of compounds of the formula (Ic) by reacting compounds of formula (VIII), wherein R¹, R² and R³ are defined above, with compounds of formula (VII), wherein P¹ is defined above, in the presence of a coupling agent, followed by deprotection of compounds of formula (VII).

Compounds of formula (VII) may also be prepared according to reaction scheme 5 by reacting compounds of formula (XI) and compounds of formula (IX), wherein P¹ represents a suitable nitrogen protecting group such as Boc, to form a compound of formula (X). The reaction is suitably carried out in the presence of a coupling reagent such EDCl and HOBt, in a suitable solvent such as DCM (suitably at room temperature to reflux) and is followed by alkylation of compounds of formula (X) with an alkylating agent in a suitable solvent such as DMF (suitably at room temperature) in the presence of a base such as potassium or cesium carbonate.

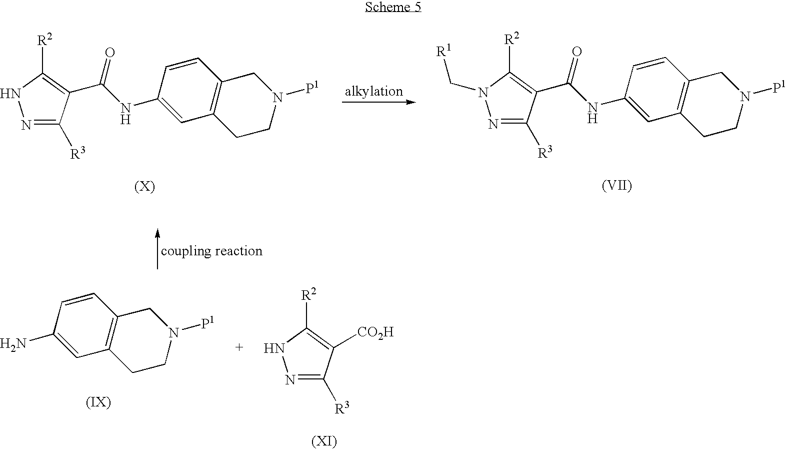

Compounds of formula (I), wherein X represents —CONH— and R⁴ represents —C₁₋₆alkyl (formula (Id)), may be prepared according to reaction scheme 6 by reacting compounds of formula (VIII) and compounds of formula (IXa) in the presence of a coupling reagent such as HATU, EDCl and/or HOBt, in a suitable solvent such as DCM (suitably at room temperature to reflux).

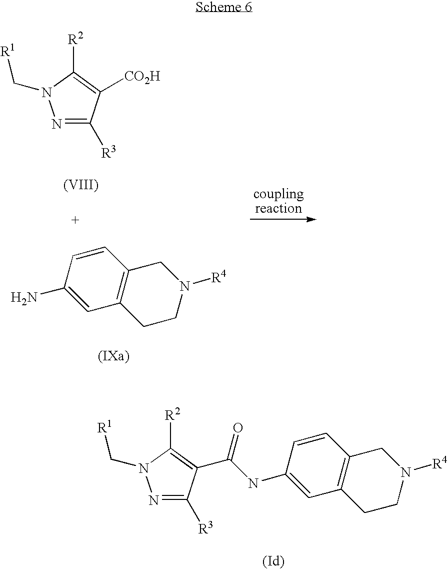

Accordingly, in one aspect the invention provides a process for the preparation of compounds of the formula (Id) by reacting compounds of formula (VIII), wherein R¹, R² and R³ are defined above, with compounds of formula (IXa), wherein R⁴ is defined above, in the presence of a coupling agent.

Compounds of formula (I), wherein X represents —NHCONH— and R⁴ represents H (formula (Ie)) may be prepared according to reaction scheme 7 by reacting compounds of formula (III) and compounds of formula (IX), wherein P¹ represents a suitable nitrogen protecting group such as Boc, to form a compound of formula (XII).

The reaction is suitably carried out in the presence of triphosgene, N,N′-carbonyldiimidazole or alkyl chloroformate in a suitable solvent such as THF (suitably at reflux), and is followed by deprotection of compounds of formula (VIII) under acidic conditions such as hydrochloric acid in a suitable solvent such as EtOAc (suitably at room temperature).

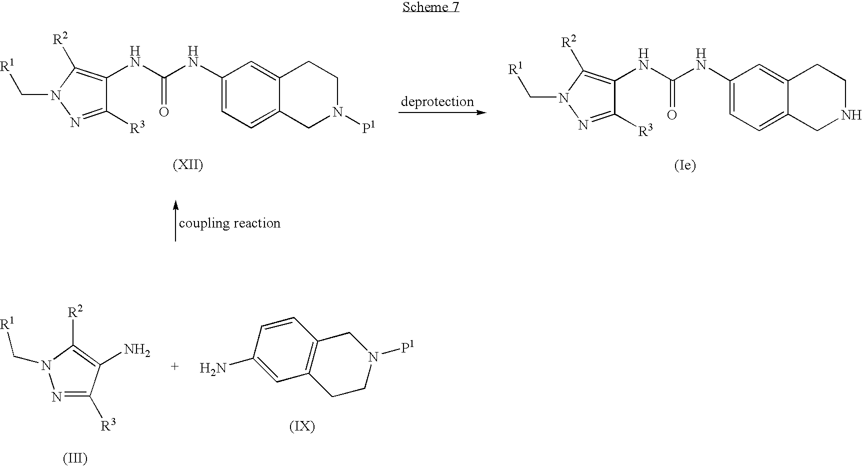

Accordingly, in one aspect the invention provides a process for the preparation of compounds of the formula (Ie) by reacting compounds of formula (III), wherein R¹, R² and R³ are defined above, with compounds of formula (IX), wherein P¹ is defined above, in the presence of a coupling agent. Followed by deprotection of compounds of formula (XII) under acidic conditions.

Compounds of formula (I), wherein X represents —NHCONH— and R⁴ represents C₁₋₆alkyl (formula (If)), may be prepared according to reaction scheme 8 by reacting compounds of formula (III) and compounds of formula (IXa) in the presence of triphosgene, N,N′-carbonyldiimidazole or a chloroformate in a suitable solvent such as THF (suitably at reflux temperature).

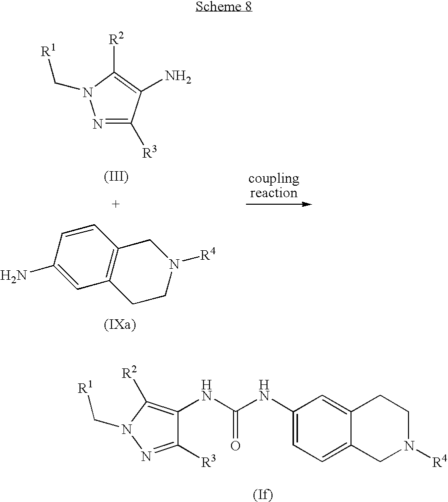

Accordingly, in one aspect the invention provides a process for the preparation of compounds of the formula (If) by reacting compounds of formula (III), wherein R¹, R² and R³ are defined above, with compounds of formula (IXa), wherein R⁴ is defined above.

Compounds of formula (I), wherein X represents —NHCO—, —CONH— or —NHCONH— and R⁴ represents —C(═O)C₁₋₆alkyl or —C(═O)C₃₋₆cycloalkyl (formula Ig), may be prepared according to reaction scheme 9 by reacting compounds of formula (Ia) with a compound of formula R⁴—Cl in the presence of a base such as pyridine, in a suitable solvent such as THF (suitably at room temperature).

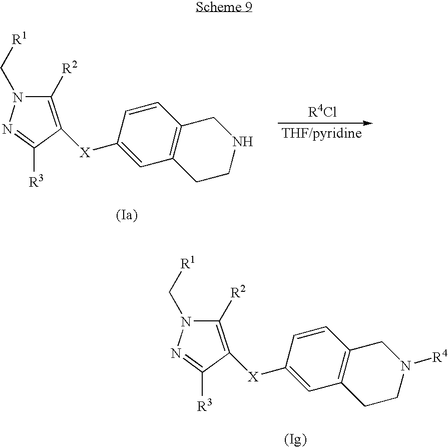

Accordingly, in one aspect the invention provides a process for the preparation of compounds of the formula (Ig) by reacting compounds of formula (Ia), wherein R¹, R² and R³ are defined above, with compounds of formula R⁴—Cl.

Compounds of formula (III) may be prepared according to reaction scheme 10 by reacting compounds of formula (XIII) with compounds of formula (XIV), wherein L¹ represents a leaving group such as a halide, in the presence of a base such as potassium carbonate in a suitable solvent such as DMF (suitably at reflux temperature), followed by reduction of the nitro group of compound of formula (XV) in the presence of a reducing agent such as SnCl₂.2H₂O in a suitable solvent such as ethanol (suitably at reflux temperature).

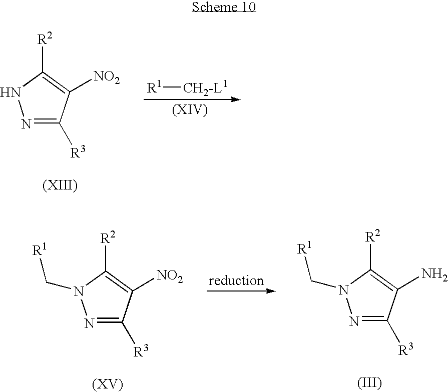

Compounds of formula (VIII) may be prepared according to reaction scheme 11 by reacting compounds of formula (XVI) with compounds of formula (XIV), wherein L² represents a leaving group such as a halide, in the presence of a base such as potassium carbonate in a suitable solvent such as DMF (suitably at reflux temperature), followed by saponification of compound of formula (XVII) with a base such as sodium hydroxide in a suitable solvent such as ethanol (suitably at reflux temperature).

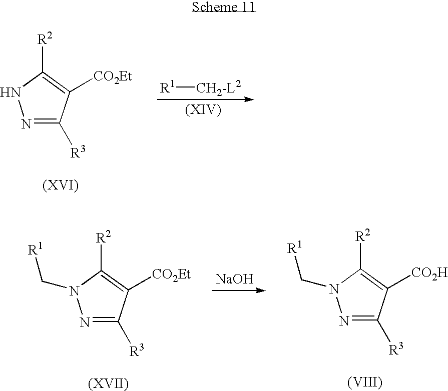

Compounds of formula (IVa) may be prepared according to reaction scheme 12 by a reductive amination of compounds of formula (XVIII) with an aldehyde in the presence of a reducing agent such as sodium triacetoxy borohydride, followed by saponification of compounds of formula (XIX) in the presence of a base such as sodium hydroxide in a suitable solvent such as ethanol (suitably at reflux).

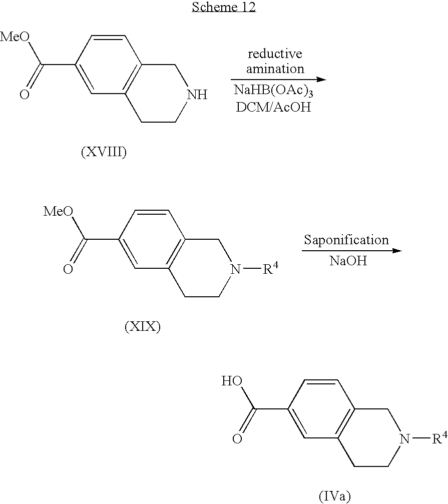

Compounds of formula (IXa) may be prepared according to reaction scheme 13 by a reductive amination of compounds of formula (XX) with an aldehyde in the presence of a reducing agent such as sodium triacetoxy borohydride or by alkylation with appropriate halide followed by reduction of the nitro group of compounds of formula (XXI).

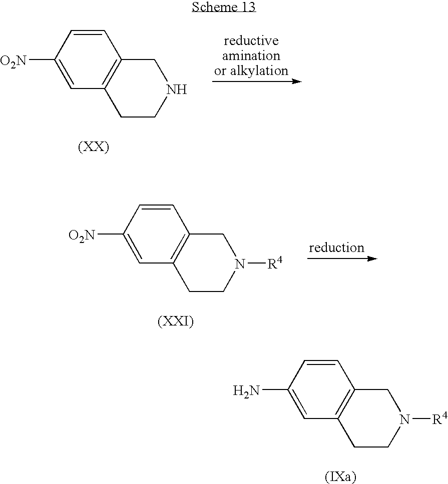

Compounds of formula (I), wherein R⁴ represents —CO₂C₁₋₆alkyl (compounds of formula (II), (V), (VII), (X) and (XII)), may be prepared according to the reaction schemes given above, wherein P¹ represents —CO₂C₁₋₆alkyl.

Compounds of formula (IV), (VI), (IX), (XI), (XIII), (XVI), (XVIII) and (XX) are commercially available or may be prepared by methods known in the literature or processes known to those skilled in the art.

Further details for the preparation of compounds of formula (I) are found in the examples section hereinafter.

The compounds of the invention may be prepared singly or as compound libraries comprising at least 2, for example 5 to 1,000 compounds, and more preferably 10 to 100 compounds. Libraries of compounds of the invention may be prepared by a combinatorial ‘split and mix’ approach or by multiple parallel synthesis using either solution phase or solid phase chemistry, by procedures known to those skilled in the art. Thus according to a further aspect there is provided a compound library comprising at least 2 compounds of the invention.

Those skilled in the art will appreciate that in the preparation of compounds of formula (I) and/or solvates thereof it may be necessary and/or desirable to protect one or more sensitive groups in the molecule or the appropriate intermediate to prevent undesirable side reactions. Suitable protecting groups for use according to the present invention are well known to those skilled in the art and may be used in a conventional manner. See, for example, “Protective groups in organic synthesis” by T. W. Greene and P. G. M. Wuts (John Wiley & sons 1991) or “Protecting Groups” by P. J. Kocienski (Georg Thieme Verlag 1994). Examples of suitable amino protecting groups include acyl type protecting groups (e.g. formyl, trifluoroacetyl, acetyl), aromatic urethane type protecting groups (e.g. benzyloxycarbonyl (Cbz) and substituted Cbz), aliphatic urethane protecting groups (e.g. 9-fluorenylmethoxycarbonyl (Fmoc), t-butyloxycarbonyl (Boc), isopropyloxycarbonyl, cyclohexyloxycarbonyl) and alkyl or aralkyl type protecting groups (e.g. benzyl, trityl, chlorotrityl).

Various intermediate compounds used in the above-mentioned process, including but not limited to certain compounds of formulae (II), (V), (VII), (IX), and (XII) constitute a further aspect of the present invention.

The compounds of formula (I) or pharmaceutically acceptable salt(s) thereof may also be used in combination with other therapeutic agents. The invention thus provides, in a further aspect, a combination comprising a compound of formula (I) or pharmaceutically acceptable salt thereof together with one or more further therapeutic agent(s).

Compounds of the invention may be administered in combination with other therapeutic agents. Preferred therapeutic agents are selected from the list: an inhibitor of cholesteryl ester transferase (CETP inhibitors), a HMG-CoA reductase inhibitor, a microsomal triglyceride transfer protein, a peroxisome proliferator-activated receptor activator (PPAR), a bile acid reuptake inhibitor, a cholesterol absorption inhibitor, a cholesterol synthesis inhibitor, a fibrate, niacin, an ion-exchange resin, an antioxidant, an inhibitor of AcylCoA: cholesterol acyltransferase (ACAT inhibitor), a cannabinoid 1 antagonist, a bile acid sequestrant, a corticosteroid, a vitamin D3 derivative, a retinoid, an immunomodulator, an anti androgen, a keratolytic agent, an anti-microbial, a platinum chemotherapeutic, an antimetabolite, hydroxyurea, a taxane, a mitotic disrupter, an anthracycline, dactinomycin, an alkylating agent and a cholinesterase inhibitor.

When the compound of formula (I) or pharmaceutically acceptable salt thereof is used in combination with a second therapeutic agent the dose of each compound may differ from that when the compound is used alone. Appropriate doses will be readily appreciated by those skilled in the art. It will be appreciated that the amount of a compound of the invention required for use in treatment will vary with the nature of the condition being treated and the age and the condition of the patient and will be ultimately at the discretion of the attendant physician or veterinarian.

The combinations referred to above may conveniently be presented for use in the form of a pharmaceutical formulation and thus pharmaceutical formulations comprising a combination as defined above together with at least one pharmaceutically acceptable carrier and/or excipient comprise a further aspect of the invention. The individual components of such combinations may be administered either sequentially or simultaneously in separate or combined pharmaceutical formulations by any convenient route.

When administration is sequential, either the SCD inhibitor or the second therapeutic agent may be administered first. When administration is simultaneous, the combination may be administered either in the same or different pharmaceutical composition.

When combined in the same formulation it will be appreciated that the two compounds must be stable and compatible with each other and the other components of the formulation. When formulated separately they may be provided in any convenient formulation, conveniently in such manner as are known for such compounds in the art.

The invention also includes a pharmaceutical composition comprising one or more compounds of formula (I) or pharmaceutically acceptable salt(s) in combination with one or more excipients.

The compounds of the invention may be administered in conventional dosage forms prepared by combining a compound of the invention with standard pharmaceutical carriers or diluents according to conventional procedures well known in the art. These procedures may involve mixing, granulating and compressing or dissolving the ingredients as appropriate to the desired preparation.

The pharmaceutical compositions of the invention may be formulated for administration by any route, and include those in a form adapted for oral, topical or parenteral administration to mammals including humans.

The compositions may be in the form of tablets, capsules, powders, granules, lozenges, creams or liquid preparations, such as oral or sterile parenteral solutions or suspensions.

The topical formulations of the present invention may be presented as, for instance, dispersions, lotions, creams, gels, pastes, powders, aerosol sprays, syrups or ointments on sponges or cotton applicators, and solutions or suspensions in an aqueous liquid, non-aqueous liquid, oil-in-water emulsion, or water-in-oil liquid emulsion.

Creams, lotions, or ointments, may be prepared as rinse-off or leave-on products, as well as two stage treatment products for use with other skin cleansing or managing compositions. The compositions can be administered as a rinse-off product in a higher concentration form, such as a gel, and then a leave-on product in a lower concentration to avoid irritation of the skin. Each of these forms is well understood by those of ordinary skill in the art, such that dosages may be easily prepared to incorporate the pharmaceutical composition of the invention.

Ointments are hydrocarbon-based semisolid formulations containing dissolved or suspended drugs. Creams and lotions are semi-solid emulsion systems and the term is applied both to water/oil or oil/water. Gel formulations are semi-solid systems in which a liquid phase is trapped in a polymeric matrix.

By way of non-limiting example, the ointments may contain one or more hydrophobic carriers selected from, for example, white soft paraffin or other mineral waxes, liquid paraffin, non-mineral waxes, long chain alcohols, long chain acids and silicones. The ointment may contain in addition to the hydrophobic carriers some hydrophillic carriers selected from, for example, propylene glycol and polyethylene glycol in combination with an appropriate surfactant/co-surfactant system. The carrier compositions of the creams or lotions are typically based on water, white soft paraffin and an appropriate surfactant/co-surfactant system, in combination with other carriers/components selected from, for example, propylene glycol, butylene glycol glycerinemonostearate, PEG-glycerinemonostearate, esters such as C₁₂₋₁₅ alkyl benzoate, liquid paraffin, non-mineral waxes, long chain alcohols, long chain acids silicones, non-silicone polymers. The gels may by way of example be formulated using isopropyl alcohol or ethyl alcohol, propylene glycol and water with a gelling agent such as hydroxyethyl cellulose, suitably in combination with minor components, for example one or more of butylene glycol and a wetting agent such as a poloxamer.

An ointment, cream, lotion, gel, and the like, can further comprise a moisturizing agent. The moisturizing agent can be a hydrophobic moisturizing agent such as ceramide, borage oil, tocopherol, tocopherol linoleate, dimethicone or a mixture thereof or a hydrophilic moisturizing agent such as glycerine, hyaluronic acid, sodium peroxylinecarbolic acid, wheat protein, hair keratin amino acids, or a mixture thereof.

The compositions according to the invention may also comprise conventional additives and adjuvants for dermatological applications, such as preservatives, acids or bases used as pH buffer excipients and antioxidants.

The present invention encompasses administration via a transdermal patch or other forms of transdermal administration. Suitable formulations for transdermal administration are known in the art, and may be employed in the methods of the present invention. For example, suitable transdermal patch formulations for the administration of a pharmaceutical compound are described in, for example, U.S. Pat. No. 4,460,372 to Campbell et al., U.S. Pat. No. 4,573,996 to Kwiatek et al., U.S. Pat. No. 4,624,665 to Nuwayser, U.S. Pat. No. 4,722,941 to Eckert et al., and U.S. Pat. No. 5,223,261 to Nelson et al.

One suitable type of transdermal patch for use in the methods of the present invention encompasses a suitable transdermal patch includes a backing layer which is non-permeable, a permeable surface layer, an adhesive layer substantially continuously coating the permeable surface layer, and a reservoir located or sandwiched between the backing layer and the permeable surface layer such that the backing layer extends around the sides of the reservoir and is joined to the permeable surface layer at the edges of the permeable surface layer. The reservoir contains a compound of formula (I) or pharmaceutically acceptable salt thereof, alone or in combination, and is in fluid contact with the permeable surface layer. The transdermal patch is adhered to the skin by the adhesive layer on the permeable surface layer, such that the permeable surface layer is in substantially continuous contact with the skin when the transdermal patch is adhered to the skin. While the transdermal patch is adhered to the skin of the subject, the compound of formula (I) or pharmaceutically acceptable salt thereof contained in the reservoir of the transdermal patch is transferred via the permeable surface layer, from the reservoir, through the adhesive layer, and to the skin of the patient. The transdermal patch may optionally also include one or more penetration-enhancing agents in the reservoir that enhance the penetration of the compound of formula (I) or pharmaceutically acceptable salt thereof through the skin.

Examples of suitable materials which may comprise the backing layer are well known in the art of transdermal patch delivery, and any conventional backing layer material may be employed in the transdermal patch of the instant invention.

Suitable penetration-enhancing agents are well known in the art as well. Examples of conventional penetration-enhancing agents include alkanols such as ethanol, hexanol, cyclohexanol, and the like, hydrocarbons such as hexane, cyclohexane, isopropylbenzene; aldehydes and ketones such as cyclohexanone, acetamide, N,N-di(lower alkyl)acetamides such as N,N-diethylacetamide, N,N-dimethyl acetamide, N-(2-hydroxyethyl)acetamide, esters such as N,N-di-lower alkyl sulfoxides; essential oils such as propylene glycol, glycerine, glycerol monolaurate, isopropyl myristate, and ethyl oleate, salicylates, and mixtures of any of the above.

Tablets and capsules for oral administration may be in unit dose presentation form, and may contain conventional excipients such as binding agents, for example syrup, acacia, gelatin, sorbitol, tragacanth, or polyvinylpyrrolidone; fillers, for example lactose, sugar, maize-starch, calcium phosphate, sorbitol or glycine; tabletting lubricants, for example magnesium stearate, talc, polyethylene glycol or silica; disintegrants, for example potato starch; or acceptable wetting agents such as sodium lauryl sulphate. The tablets may be coated according to methods well known in normal pharmaceutical practice. Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives, such as suspending agents, for example sorbitol, methyl cellulose, glucose syrup, gelatin, hydroxyethyl cellulose, carboxymethyl cellulose, aluminium stearate gel or hydrogenated edible fats, emulsifying agents, for example lecithin, sorbitan monooleate, or acacia; non-aqueous vehicles (which may include edible oils), for example almond oil, oily esters such as glycerine, propylene glycol, or ethyl alcohol; preservatives, for example methyl or propyl p-hydroxybenzoate or sorbic acid, and, if desired, conventional flavouring or colouring agents.

Preparations for oral administration may be suitably formulated to give controlled/extended release of the active compound.

Suppositories will contain conventional suppository bases, e.g. cocoa-butter or other glyceride.

For parenteral administration, fluid unit dosage forms are prepared utilising the compound and a sterile vehicle, water being preferred. The compound, depending on the vehicle and concentration used, can be either suspended or dissolved in the vehicle. In preparing solutions the compound can be dissolved in water for injection and filter sterilised before filling into a suitable vial or ampoule and sealing.

Advantageously, agents such as a local anaesthetic, preservative and buffering agents can be dissolved in the vehicle. To enhance the stability, the composition can be frozen after filling into the vial and the water removed under vacuum. The dry lyophilised powder is then sealed in the vial and an accompanying vial of water for injection may be supplied to reconstitute the liquid prior to use. Parenteral suspensions are prepared in substantially the same manner except that the compound is suspended in the vehicle instead of being dissolved and sterilisation cannot be accomplished by filtration. The compound can be sterilised by exposure to ethylene oxide before suspending in the sterile vehicle. Advantageously, a surfactant or wetting agent is included in the composition to facilitate uniform distribution of the compound.

The compositions may contain from 0.1% by weight, preferably from 10-60% by weight, of the active ingredient, depending on the method of administration. Where the compositions comprise dosage units, each unit will preferably contain from 50-500 mg of the active ingredient. The dosage as employed for adult human treatment will preferably range from 100 to 3000 mg per day, for instance 1500 mg per day depending on the route and frequency of administration. Such a dosage corresponds to 1.5 to 50 mg/kg per day. Suitably the dosage is from 5 to 20 mg/kg per day.

It will be recognised by one of skill in the art that the optimal quantity and spacing of individual dosages of a compound of the invention will be determined by the nature and extent of the condition being treated, the form, route and site of administration, and the particular mammal being treated, and that such optimums can be determined by conventional techniques. It will also be appreciated by one of skill in the art that the optimal course of treatment, i.e., the number of doses of a compound of the invention given per day for a defined number of days, can be ascertained by those skilled in the art using conventional course of treatment determination tests.

The invention also extends to novel intermediates disclosed herein, used in the preparation of compounds of formula (I)

### DEFINITIONS

EtOAc Ethyl acetate
Boc Tertbutyloxy carbonyl
CCl₄ Carbon tetrachloride
DCM Dichloromethane
DIEA Diisopropyl ethyl amine
DMF Dimethyl formamide
Et₃N Triethylamine
EtOH Ethanol
Fmoc 9-Fluorenylmethoxycarbonyl
HATU O-(7-Azobenzotriazol-1-yl)-1,1,3,3-tetramethyluronium hexafluorophosphate
HCl Hydrochloric acid
HOBt 1-Hydroxybenzotriazole
Me Methyl
MeCN Acetonitrile
MeOH Methanol
NaHB(OAc)₃ Triacetoxy sodium borohydride
NaOH Sodium hydroxide
NH₂NH₂ Hydrazine

Regardless of how the preparation of compounds are represented in the present specification no inference can be drawn that particular batches (or mixtures of two or more batches) of intermediates were used in the next stage of the preparation. The examples and intermediates are intended to illustrate the synthetic routes suitable for preparation of the same, to assist the skilled persons understanding of the present invention.

Where reference is made to the use of a “similar” procedure, as will be appreciated by those skilled in the art, such a procedure may involve minor variation, for example reaction temperature, reagent/solvent amount, reaction time, work-up conditions or chromatographic purification conditions.

### Analytical Methods LC-MS

Analytical HPLC was conducted on a X-terra MS C18 column (2.5 μm 3×30 mm id) eluting with 0.01M ammonium acetate in water (solvent A) and 100% acetonitrile using the following elution gradient: 0 to 4 minutes, 5 to 100% B; 4 to 5 minutes, 100% B at a flow-rate of 1.1 mL/min with a temperature of 40° C.

The mass spectra (MS) were recorded on a micromass ZQ-LC mass spectrometer using electrospray positive ionisation [ES+ve to give MH⁺ molecular ion] or electrospray negative ionisation [ES−ve to give (M−H)⁻ molecular ion] modes.

### Analytical Methods LC-HRMS

Analytical HPLC was conducted on an Uptisphere-hsc column (3 μm 30×3 mm id) eluting with 0.01M ammonium acetate in water (solvent A) and 100% acetonitrile (solvent B) using the following elution gradient: 0 to 0.5 minutes, 5% B; 0.5 to 3.5 minutes, 5 to 100% B; 3.5 to 4 minutes, 100% B; 4 to 4.5 minutes, 100 to 5% B; 4.5 to 5.5 minutes, 5% B at a flow-rate of 1.3 mL/min with a temperature of 40° C.

The mass spectra (MS) were recorded on a micromass LCT, mass spectrometer using electrospray positive ionisation [ES+ve to give MH⁺ molecular ion] or electrospray negative ionisation [ES−ve to give (M−H)⁻ molecular ion] modes.

### Analytical Method GC-MS

Analytical GC was conducted on a DB-1 ms column (Agilent Technologies), 0.1 μm 10 m×0.1 mm id) eluting with an Helium flow of 0.5 ml/min and pressure at 3.4 bar and with a gradient temperature: 0 to 0.35 min, 100° C.; 0.35 min to 6 min, 100° C. to 250° C. (ramp of 80° C./min).

The mass spectra (MS) were recorded on a Agilent Technologies G5973 mass spectrometer using electronic impact ionisation.

The following non-limiting examples illustrate the present invention.

### Intermediate 1: 1-[(3,4-Dichlorophenyl)methyl]-4-nitro-1H-pyrazole

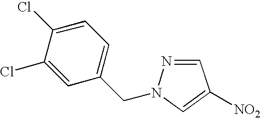

To a solution of 4-nitro-1H-pyrazole (0.618 g, 5.5 mmol) and potassium carbonate (0.906 g, 6.56 mmol) in DMF (10 mL) was added dropwise a solution of 1,2-dichloro-4-(chloromethyl)benzene (1.07 g, 5.5 mmol) in DMF (5 mL) and the reaction mixture was stirred at room temperature for 2 hours. After evaporation of the solvent under reduced pressure, the residue was diluted with water and extracted with DCM. The combined extracts were washed with brine, dried over Na₂SO₄, filtered and evaporated under reduced pressure. The residue was purified by flash column chromatography eluting with DCM/MeOH:99/1 to give the title compound as a white solid (1.3 g, 88%).

LC/MS: m/z 272 (M+H)⁺, Rt: 3.27 min.

### Intermediate 2: Ethyl 1-[(3,4-dichlorophenyl)methyl]-1H-pyrazole-4-carboxylate

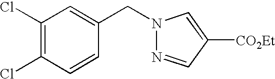

To a solution of ethyl 1H-pyrazole-4-carboxylate (0.475 mg, 3.4 mmol) and potassium carbonate (0.56 g, 4.08 mmol) in DMF (20 mL) was added dropwise 1,2-dichloro-4-(bromomethyl)benzene (0.98 g, 4.08 mmol) and the reaction mixture was stirred at room temperature overnight. After evaporation of the solvent under reduced pressure, the residue was diluted with water and extracted with DCM. The combined extracts were washed with brine, dried over Na₂SO₄, filtered and evaporated under reduced pressure. The residue was purified by flash column chromatography eluting with DCM/EtOAc:92/8 to give the title compound as a white solid (0.92 g, 91%).

LC/MS: m/z 299 (M+H)⁺, Rt: 3.44 min.

The following compounds were similarly prepared by a method analogous to that described for Intermediate 2.

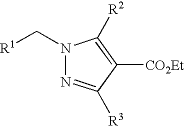

TABLE 1

| Intermediate No. | R¹ | R² | R³ | From | Physical data |
|---|---|---|---|---|---|
| 3 Ethyl 1-[(3,4- dichlorophenyl)methyl]- 5-methyl-1H-pyrazole-4- carboxylate | 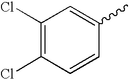 | Me | H | Ethyl 5-methyl- 1H-pyrazole-4- carboxylate | LC/MS: m/z 313 (M + H)⁺, Rt: 3.49 min |
| 4 Ethyl 1-[(3,4- dichlorophenyl)methyl]- 3-methyl-1H-pyrazole-4- carboxylate | 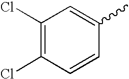 | H | Me | Ethyl 5-methyl- 1H-pyrazole-4- carboxylate | LC/MS: m/z 313 (M + H)⁺, Rt: 3.45 min |
| 5 Ethyl 1-(1- naphthalenylmethyl)- 1H-pyrazole-4- carboxylate | 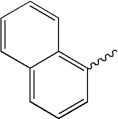 | H | H | Ethyl 1H- pyrazole-4- carboxylate | LC/MS: m/z 281 (M + H)⁺, Rt: 3.22 min |
| 6 Ethyl 1-{[3- (trifluoromethyl)phenyl] methyl}-1H-pyrazole-4- carboxylate | 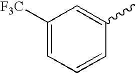 | H | H | Ethyl 1H- pyrazole-4- carboxylate | LC/MS: m/z 299 (M + H)⁺, Rt: 3.27 min |

### Intermediate 7: 1-[(3,4-Dichlorophenyl)methyl]-1H-pyrazol-4-amine

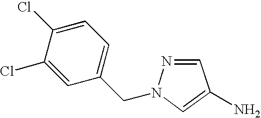

To a solution of 1-[(3,4-dichlorophenyl)methyl]-4-nitro-1H-pyrazole (Intermediate 1) (1.3 g, 4.79 mmol) in EtOH (35 mL) was added SnCl₂.2H₂O (5.39 g, 23.98 mmol) and the mixture was stirred at reflux overnight. After cooling at room temperature, the mixture was poured into water and basified with a saturated solution of NaHCO₃. The aqueous phase was extracted with EtOAc and the organic phase was washed with brine, dried over Na₂SO₄, filtered and evaporated under reduced pressure. The residue was purified by flash column chromatography eluting with CH₂Cl₂/MeOH:95/5 to give the title compound as a colorless oil (0.46 g, 40%).

LC/MS: m/z 242 (M+H)⁺, Rt: 2.53 min.

### Intermediate 8: 1-[(3,4-Dichlorophenyl)methyl]-1H-pyrazole-4-carboxylic acid

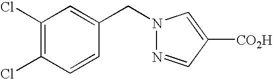

To a solution of ethyl 1-[(3,4-dichlorophenyl)methyl]-1H-pyrazole-4-carboxylate (Intermediate 2) (0.92 g, 3.08 mmol) in ethanol (20 mL) was added a 1N sodium hydroxide solution (12 mL, 12 mmol) and the reaction mixture was stirred at reflux for 2 hours. Ethanol was evaporated under reduced pressure and the residue was treated with a 1N hydrochloric acid solution. The precipitate obtained was filtered, washed with water and dried to give the title compound as a white solid (790 mg, 95%).

LC/MS: m/z 269 (M−H)⁺, Rt: 2.17 min.

The following compounds were similarly prepared by a method analogous to that described for Intermediate 8.

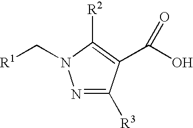

TABLE 2

| | | | | | Physical |
|---|---|---|---|---|---|
| Intermediate No. | R¹ | R² | R³ | From | data |
| 9 1-[(3,4- Dichlorophenyl)methyl]- 5-methyl-1H-pyrazole-4- carboxylic acid | 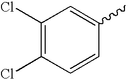 | Me | H | Ethyl 1-[(3,4- dichlorophenyl) methyl]-5-methyl- 1H-pyrazole-4- carboxylate (Intermediate 3) | LC/MS: m/z 285 (M + H)⁺, Rt: 2.14 min |
| 10 1-[(3,4- Dichlorophenyl)methyl]- 3-methyl-1H-pyrazole-4- carboxylic acid | 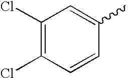 | H | Me | Ethyl 1-[(3,4- dichlorophenyl) methyl]-3-methyl- 1H-pyrazole-4- carboxylate (Intermediate 4) | LC/MS: m/z 285 (M + H)⁺, Rt: 2.11 min |
| 11 1-(1- Naphthalenylmethyl)- 1H-pyrazole-4- carboxylic acid | 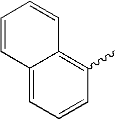 | H | H | Ethyl 1-(1- naphthalenylmethyl)- 1H-pyrazole-4- carboxylate (Intermediate 5) | LC/MS: m/z 251 (M − H)⁻, Rt: 2.18 min |
| 12 1-{[3- (Trifluoromethyl)phenyl] methyl}-1H-pyrazole-4- carboxylic acid | 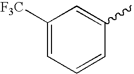 | H | H | Ethyl 1-{[3- (trifluoromethyl)phe- nyl]methyl}-1H- pyrazole-4- carboxylate (Intermediate 6) | LC/MS: m/z 269 (M − H)⁻, Rt: 1.92 min |

### Intermediate 13: 1,1-Dimethylethyl 6-({[1-(1-naphthalenylmethyl)-1H-pyrazol-4-yl]amino}carbonyl)-3,4-dihydro-2(1H)-isoquinolinecarboxylate

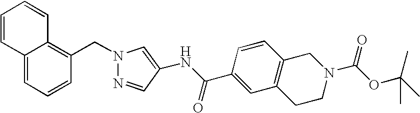

To a solution of 2-{[(1,1-dimethylethyl)oxy]carbonyl}-1,2,3,4-tetrahydro-6-isoquinoline carboxylic acid (138 mg, 0.5 mmol), N-(3-dimethylaminopropyl)-N′-ethylcarbodiimide hydrochloride (114 mg, 0.6 mmol), 1-hydroxybenzotriazole hydrate (81 mg, 0.6 mmol) and triethylamine (60 mg, 0.6 mmol) in DCM (10 mL) was added 1-(1-naphthalenylmethyl)-1H-pyrazol-4-amine (111 mg, 0.5 mmol) and the reaction mixture was stirred at room temperature overnight. The organic phase was diluted with an additional volume of DCM then washed with a saturated sodium hydrogeno carbonate solution, with brine, dried over Na₂SO₄, filtered and evaporated under reduced pressure. The residue was purified by flash column chromatography eluting with DCM/MeOH:97/3 to give the title compound as an orange oil (180 mg, 81%).

LC/MS: m/z 483 (M+H)⁺, Rt: 3.53 min.

The following compounds were similarly prepared by a method analogous to that described for Intermediate 13.

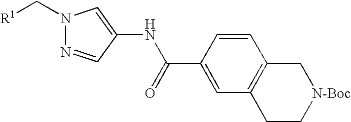

TABLE 3

| Intermediate No. | R¹ | From | Physical data |
|---|---|---|---|
| 14 1,1-Dimethylethyl 6-[({1-[(2,4- dichlorophenyl)methyl]-1H- pyrazol-4-yl}amino)carbonyl]- 3,4-dihydro-2(1H)- isoquinolinecarboxylate | 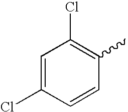 | 1-[(2,4- dichlorophenyl) methyl]-1H- pyrazol-4-amine | LC/MS: m/z 501 (M + H)⁺, Rt: 3.60 min |
| 15 1,1-Dimethylethyl 6-[({1-[(2- methylphenyl)methyl]-1H- pyrazol-4-yl}amino)carbonyl]- 3,4-dihydro-2(1H)- isoquinolinecarboxylate | 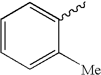 | 1-[(2- methylphenyl) methyl]-1H- pyrazol-4-amine | LC/MS: m/z 447 (M + H)⁺, Rt: 3.38 min |

The following compounds were similarly prepared by a method analogous to that described for Intermediate 13 but were used directly in the next step without purification.

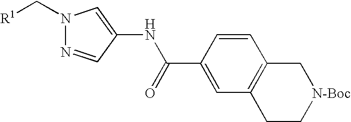

TABLE 4

| Intermediate No. | R¹ | From |
|---|---|---|
| 16 1,1-Dimethylethyl 6-[({1-[(3- chlorophenyl)methyl]-1H-pyrazol-4- yl}amino)carbonyl]-3,4-dihydro-2(1H)- isoquinolinecarboxylate | 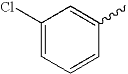 | 1-[(3- chlorophenyl)methyl]-1H- pyrazol-4-amine |
| 17 1,1-dimethylethyl 6-[({1-[(2- chlorophenyl)methyl]-1H-pyrazol-4- yl}amino)carbonyl]-3,4-dihydro-2(1H)- isoquinolinecarboxylate | 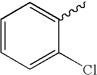 | 1-[(2- chlorophenyl)methyl]-1H- pyrazol-4-amine |
| 18 1,1-dimethylethyl 6-{[(1-{[3- (methyloxy)phenyl]methyl}-1H- pyrazol-4-yl)amino]carbonyl}-3,4- dihydro-2(1H)-isoquinolinecarboxylate | 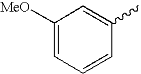 | 1-{[3- (methyloxy)phenyl]methyl}- 1H-pyrazol-4-amine |
| 19 1,1-dimethylethyl 6-[({1-[(3- fluorophenyl)methyl]-1H-pyrazol-4- yl}amino)carbonyl]-3,4-dihydro-2(1H)- isoquinolinecarboxylate | 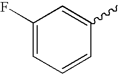 | 1-[(3- fluorophenyl)methyl]-1H- pyrazol-4-amine |
| 20 1,1-dimethylethyl 6-{[(1-{[3- (trifluoromethyl)phenyl]methyl}-1H- pyrazol-4-yl)amino]carbonyl}-3,4- dihydro-2(1H)-isoquinolinecarboxylate | 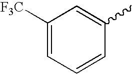 | 1-{[3- (trifluoromethyl)phenyl]meth- yl}-1H-pyrazol-4-amine |
| 21 1,1-dimethylethyl 6-[({1-[(4- chlorophenyl)methyl]-1H-pyrazol-4- yl}amino)carbonyl]-3,4-dihydro-2(1H)- isoquinolinecarboxylate | 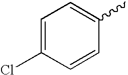 | 1-[(4- chlorophenyl)methyl]-1H- pyrazol-4-amine |
| 22 1,1-dimethylethyl 6-[({1-[(3- methylphenyl)methyl]-1H-pyrazol-4- yl}amino)carbonyl]-3,4-dihydro-2(1H)- isoquinolinecarboxylate | 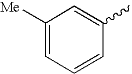 | 1-[(3- methylphenyl)methyl]-1H- pyrazol-4-amine |
| 23 1,1-dimethylethyl 6-[({1-[(4- methylphenyl)methyl]-1H-pyrazol-4- yl}amino)carbonyl]-3,4-dihydro-2(1H)- isoquinolinecarboxylate | 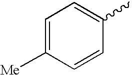 | 1-[(4- methylphenyl)methyl]-1H- pyrazol-4-amine |
| 24 1,1-dimethylethyl 6-[({1-[(4- fluorophenyl)methyl]-1H-pyrazol-4- yl}amino)carbonyl]-3,4-dihydro-2(1H)- isoquinolinecarboxylate | 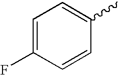 | 1-[(4- fluorophenyl)methyl]-1H- pyrazol-4-amine |

### Intermediate 25: 1,1-Dimethylethyl 6-[(1H-pyrazol-4-ylamino)carbonyl]-3,4-dihydro-2(1H)-isoquinolinecarboxylate

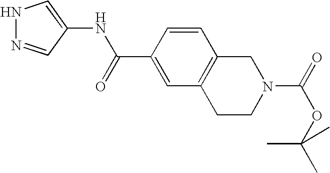

To a solution of 1H-pyrazol-4-amine (1.87 g, 22.5 mmol), N-(3-dimethylaminopropyl)-N′-ethylcarbodiimide hydrochloride (5.18 g, 27 mmol), 1-hydroxybenzotriazole hydrate (3.65 g, 27 mmol) and triethylamine (6.3 mL, 45 mmol) in DMF was added 2-{[(1,1-dimethylethyl)oxy]carbonyl}-1,2,3,4-tetrahydro-6-isoquinolinecarboxylic acid (5.62 g, 20.3 mmol) and the reaction mixture was stirred at room temperature for 16 hours. The volatiles were removed under reduced pressure, the residue was dissolved in MeOH and potassium hydroxide (5.04 g, 90 mmol), water (100 mL) was added and the mixture was heated at 50° C. for 15 min. The MeOH was evaporated under reduced pressure and the solid which crystallized was filtered and recrystallized from acetonitrile to give the title compound as a solid (3.7 g, 48%).

LC/MS: m/z 341 (M−H)⁺, Rt: 2.80 min.

### Intermediate 26: 1,1-Dimethylethyl 6-({[1-(cyclohexylmethyl)-1H-pyrazol-4-yl]amino}carbonyl)-3,4-dihydro-2(1H)-isoquinolinecarboxylate

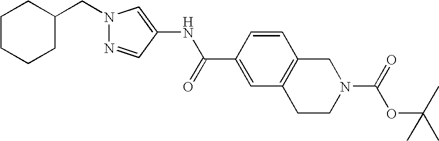

To a stirred suspension of 1,1-dimethylethyl 6-[(1H-pyrazol-4-ylamino)carbonyl]-3,4-dihydro-2(1H)-isoquinolinecarboxylate (Intermediate 25) (100 mg, 0.29 mmol) and potassium carbonate (49 mg, 0.35 mmol) in DMF (5 mL) was added dropwise (bromomethyl)cyclohexane (62 mg, 0.35 mmol) and the mixture was heated at 80° C. for 24 hours. (Bromomethyl)cyclohexane (62 mg, 0.35 mmol) and potassium carbonate (49 mg, 0.35 mmol) was added and the mixture was heated at 80° C. for 24 hours. The volatiles were removed under reduced pressure, the residue was dissolved in DCM and the organic phase was washed with brine, dried over Na₂SO₄, filtered and evaporated to dryness. The residue was purified by flash column chromatography eluting with DCM/MeOH:94/6 to give the title compound as a pale yellow solid (50 mg, 39%).

LC/MS: m/z 439 (M+H)⁺, Rt: 3.63 min.

The following compound was similarly prepared by a method analogous to that described for Intermediate 26:

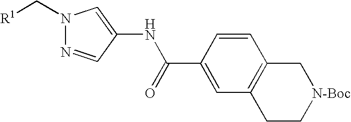

TABLE 5

| Intermediate No. | R¹ | From | Physical data |
|---|---|---|---|
| 27 1,1-dimethylethyl 6-{[(1-{[4- (trifluoromethyl)phenyl] methyl}-1H-pyrazol-4- yl)amino]carbonyl}-3,4- dihydro-2(1H)- isoquinolinecarboxylate | 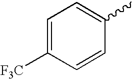 | 1,1-dimethylethyl 6- [(1H-pyrazol-4- ylamino)carbonyl]-3,4- dihydro-2(1H)- isoquinolinecarboxylate (Intermediate 25) | LC/MS: m/z 501 (M + H)⁺, Rt: 3.57 min |

### Intermediate 28: 1,1-Dimethylethyl 6-[({1-[(3,4-dichlorophenyl)methyl]-1H-pyrazol-4-yl}carbonyl)amino]-3,4-dihydro-2(1H)-isoquinolinecarboxylate

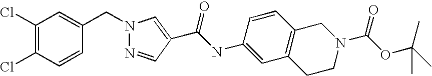

To a solution of 1-[(3,4-dichlorophenyl)methyl]-1H-pyrazole-4-carboxylic acid (Intermediate 8) (105 mg, 0.39 mmol), N-(3-dimethylaminopropyl)-N′-ethylcarbodiimide hydrochloride (75 mg, 0.39 mmol), 1-hydroxybenzotriazole hydrate (52 mg, 0.39 mmol) and triethylamine (90 μL, 0.64 mmol) in DCM (5 mL) was added 1,1-dimethylethyl 6-amino-3,4-dihydro-2(1H)-isoquinolinecarboxylate (80 mg, 0.32 mmol) and the reaction mixture was stirred at room temperature for 48 hours. The organic phase was then washed with a 1N sodium hydroxide solution, with brine, dried over Na₂SO₄, filtered and evaporated under reduced pressure. The residue was purified by flash column chromatography eluting with DCM/MeOH:95/5 to give the title compound as a yellow oil (120 mg, 75%).

LC/MS: m/z 501 (M+H)⁺, Rt: 3.75 min.

The following compounds were similarly prepared by a method analogous to that described for Intermediate 28.

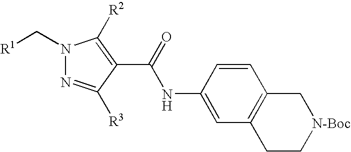

TABLE 6

| | | | | | Physical |
|---|---|---|---|---|---|
| Intermediate No | R¹ | R² | R³ | From | data |
| 29 1,1-Dimethylethyl 6-({[1- (phenylmethyl)-1H-pyrazol- 4-yl]carbonyl}amino)-3,4- dihydro-2(1H)- isoquinolinecarboxylate | 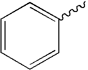 | H | H | 1-(phenylmethyl)- 1H-pyrazole-4- carboxylic acid | LC/MS: m/z 433 (M + H)⁺, Rt: 3.41 min |
| 30 1,1-Dimethylethyl 6-[({1- [(3,4-dichlorophenyl)methyl]- 5-methyl-1H-pyrazol-4- yl}carbonyl)amino]-3,4- dihydro-2(1H)- isoquinolinecarboxylate | 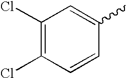 | Me | H | 1-[(3,4- dichlorophenyl) methyl]-5-methyl- 1H-pyrazole-4- carboxylic acid (Intermediate 9) | LC/MS: m/z 515 (M + H)⁺, Rt: 3.88 min |
| 31 1,1-Dimethylethyl 6-[({1- [(3,4-dichlorophenyl)methyl]- 3-methyl-1H-pyrazol-4- yl}carbonyl)amino]-3,4- dihydro-2(1H)- isoquinolinecarboxylate | 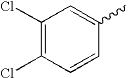 | H | Me | 1-[(3,4- dichlorophenyl) methyl]-3-methyl- 1H-pyrazole-4- carboxylic acid (Intermediate 10) | LC/MS: m/z 515 (M + H)⁺, Rt: 3.72 min |
| 32 1,1-Dimethylethyl 6-{[(1-{[3- (trifluoromethyl)phenyl]meth- yl}-1H-pyrazol-4- yl)carbonyl]amino}-3,4- dihydro-2(1H)- isoquinolinecarboxylate | 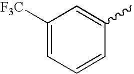 | H | H | 1-{[3- (trifluoromethyl) phenyl]methyl}- 1H-pyrazole-4- carboxylic acid (Intermediate 12) | LC/MS: m/z 499 (M − H)⁺, Rt: 3.57 min |
| 33 1,1-Dimethylethyl 6-({[1-(1- naphthalenylmethyl)-1H- pyrazol-4- yl]carbonyl}amino)-3,4- dihydro-2(1H)- isoquinolinecarboxylate | 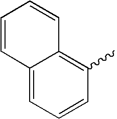 | H | H | 1-(1- naphthalenylmeth- yl)-1H-pyrazole-4- carboxylic acid (Intermediate 11) | LC/MS: m/z 483 (M + H)⁺, Rt: 3.52 min |

### Intermediate 34: 1,1-Dimethylethyl 6-[(1H-pyrazol-4-ylcarbonyl)amino]-3,4-dihydro-2(1H)-isoquinolinecarboxylate

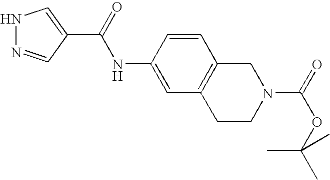

To a solution of 1H-pyrazole-4-carboxylic acid (800 mg, 7.1 mmol), N-(3-dimethylaminopropyl)-N′-ethylcarbodiimide hydrochloride (1.48 g, 7.74 mmol), 1-hydroxybenzotriazole hydrate (1.04 g, 7.74 mmol) and triethylamine (1.8 mL, 12.9 mmol) in DCM was added 1,1-dimethylethyl 6-amino-3,4-dihydro-2(1H)-isoquinolinecarboxylate (1.6 g, 6.45 mmol) and the reaction mixture was stirred at room temperature for 9 days. The organic phase was washed with 1N sodium hydroxide, dried over Na₂SO₄, filtered and evaporated under reduced pressure. The residue was purified by flash column chromatography eluting with DCM/MeOH:90/10 to give the title compound as a solid (580 mg, 24%).

LC/MS: m/z 341 (M−H)⁺, Rt: 2.70 min.

### Intermediate 35: 1,1-Dimethylethyl 6-[({1-[(4-fluorophenyl)methyl]-1H-pyrazol-4-yl}carbonyl)amino]-3,4-dihydro-2(1H)-isoquinolinecarboxylate

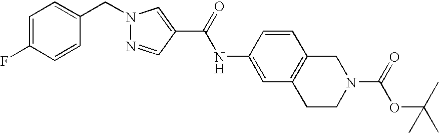

To a stirred suspension of 1,1-Dimethylethyl 6-[(1H-pyrazol-4-ylcarbonyl)amino]-3,4-dihydro-2(1H)-isoquinolinecarboxylate (Intermediate 34) (115 mg, 0.34 mmol) and cesium carbonate (131 mg, 0.40 mmol) in DMF (5 mL) was added dropwise 1-(bromomethyl)-4-fluorobenzene (71 mg, 0.37 mmol) and the mixture was heated at 80° C. for 4 days. The volatiles were removed under reduced pressure and the residue was dissolved in DCM. The organic phase was washed with water, dried over Na₂SO₄, filtered and evaporated under reduced pressure. The residue was purified by flash column chromatography eluting with DCM/MeOH:95/5 and recrystallized from diisopropyl ether to give the title compound as a white solid (80 mg, 52%).

LC/MS: m/z 451 (M+H)⁺, Rt: 3.30 min.

The following compounds were similarly prepared by a method analogous to that described for Intermediate 13.

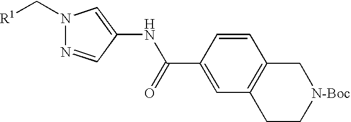

TABLE 7

| Intermediate No. | R¹ | From | Physical data |
|---|---|---|---|
| 36 1,1-dimethylethyl 6-{[(1-{[(3,4- dichlorophenyl)oxy]methyl}- 1H-pyrazol-4- yl)amino]carbonyl}-3,4- dihydro-2(1H)- isoquinolinecarboxylate | 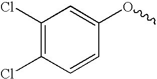 | 1-{[(3,4- dichlorophenyl) oxy]methyl}-1H- pyrazol-4-amine | LC/MS: m/z 517 (M + H)⁺, Rt: 3.74 min |
| 37 1,1-dimethylethyl 6-({[1-({[3- (trifluoromethyl) phenyl]oxy}methyl)-1H-pyrazol- 4-yl]amino}carbonyl)-3,4- dihydro-2(1H)- isoquinolinecarboxylate | 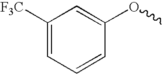 | 1-({[3- (trifluoromethyl) phenyl]oxy}methyl)- 1H-pyrazol-4- amine | LC/MS: m/z 517 (M + H)⁺, Rt: 3.68 min |
| 38 1,1-dimethylethyl 6-{[(1-{[(2,5- dichlorophenyl) oxy]methyl}-1H-pyrazol-4- yl)amino]carbonyl}-3,4- dihydro-2(1H)- isoquinolinecarboxylate | 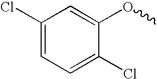 | 1-{[(2,5- dichlorophenyl) oxy]methyl}-1H- pyrazol-4-amine | LC/MS: m/z 517 (M + H)⁺, Rt: 3.65 min |
| 39 1,1-dimethylethyl 6-{[(1-{[(2,4- dichlorophenyl) oxy]methyl}-1H-pyrazol-4- yl)amino]carbonyl}-3,4- dihydro-2(1H)- isoquinolinecarboxylate | 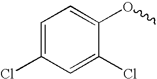 | 1-{[(2,4- dichlorophenyl) oxy]methyl}-1H- pyrazol-4-amine | LC/MS: m/z 517 (M + H)⁺, Rt: 3.65 min |
| 40 1,1-dimethylethyl 6-{[(1-{[(3- chlorophenyl) oxy]methyl}-1H-pyrazol-4- yl)amino]carbonyl}-3,4- dihydro-2(1H)- isoquinolinecarboxylate | 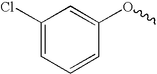 | 1-{[(3- chlorophenyl)oxy] methyl}-1H- pyrazol-4-amine | LC/MS: m/z 483 (M + H)⁺, Rt: 3.59 min |

### EXAMPLE 1

### N-{1-[(3,4-Dichlorophenyl)methyl]-1H-pyrazol-4-yl}-1,2,3,4-tetrahydro-6-isoquinolinecarboxamide hydrochloride

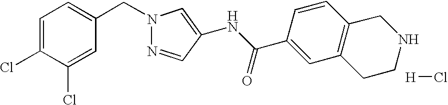

To a solution of 1,1-dimethylethyl 6-[({1-[(3,4-dichlorophenyl)methyl]-1H-pyrazol-4-yl}amino)carbonyl]-3,4-dihydro-2(1H)-isoquinolinecarboxylate (Example 24) (0.18 g, 0.36 mmol) in EtOAc (10 mL) was bubbled HCl (g) and the reaction mixture was stirred at room temperature for 1 hour. The resulting precipitate was filtered, washed with diisopropyl ether to give the title compound as a white solid (90 mg, 51%).

HRMS calculated for C₂₀H₁₈Cl₂N₄O (M+H)⁺ 401.0936, found: 401.0963, Rt: 2.43 min.

The following compounds were similarly prepared by a method analogous to that described for Example 1.

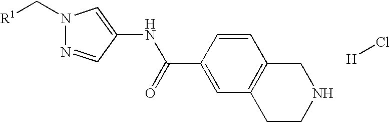

TABLE 8

| Example No. | R¹ | From | Physical data |
|---|---|---|---|
| 2 N-[1-(1- naphthalenylmethyl)-1H- pyrazol-4-yl]-1,2,3,4- tetrahydro-6- isoquinolinecarboxamide hydrochloride | 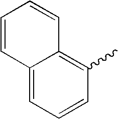 | 1,1-Dimethylethyl 6-({[1- (1-naphthalenylmethyl)- 1H-pyrazol-4- yl]amino}carbonyl)-3,4- dihydro-2(1H)- isoquinolinecarboxylate (Intermediate 13) | HRMS (M + H)⁺ calculated for C₂₄H₂₂N₄O Theo: 383.1872 Found: 383.1886 Rt: 2.25 min |
| 3 N-{1-[(2,4- dichlorophenyl)methyl]-1H- pyrazol-4-yl}-1,2,3,4- tetrahydro-6- isoquinolinecarboxamide | 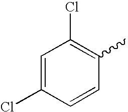 | 1,1-dimethylethyl 6-[({1- [(2,4- dichlorophenyl)methyl]- 1H-pyrazol-4- yl}amino)carbonyl]-3,4- dihydro-2(1H)- isoquinolinecarboxylate (Intermediate 14) | HRMS (M + H)⁺ calculated for C₂₀H₁₈Cl₂N₄O Theo: 401.0936 Found: 401.0935 Rt: 2.33 min |
| 4 N-{1-[(2- methylphenyl)methyl]-1H- pyrazol-4-yl}-1,2,3,4- tetrahydro-6- isoquinolinecarboxamide | 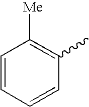 | 1,1-dimethylethyl 6-[({1- [(2-methylphenyl)methyl]- 1H-pyrazol-4- yl}amino)carbonyl]-3,4- dihydro-2(1H)- isoquinolinecarboxylate (Intermediate 15) | HRMS (M + H)⁺ calculated for C₂₁H₂₂N₄O Theo: 347.1872 Found: 347.1877 Rt: 2.11 min |
| 5 N-[1-(cyclohexylmethyl)-1H- pyrazol-4-yl]-1,2,3,4- tetrahydro-6- isoquinolinecarboxamide | 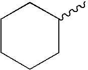 | 1,1-Dimethylethyl 6-({[1- (cyclohexylmethyl)-1H- pyrazol-4- yl]amino}carbonyl)-3,4- dihydro-2(1H)- isoquinolinecarboxylate (Intermediate 26) | HRMS (M + H)⁺ calculated for C₂₀H₂₆N₄O Theo: 339.2185 Found: 339.2181 Rt: 2.22 min |
| 6 N-(1-{[4- (trifluoromethyl)phenyl]meth- yl}-1H-pyrazol-4-yl)-1,2,3,4- tetrahydro-6- isoquinolinecarboxamide | 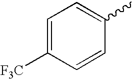 | 1,1-dimethylethyl 6-{[(1- {[4- (trifluoromethyl)phenyl]meth- yl}-1H-pyrazol-4- yl)amino]carbonyl}-3,4- dihydro-2(1H)- isoquinolinecarboxylate (Intermediate 27) | HRMS (M + H)⁺ calculated for C₂₁H₁₉F₃N₄O Theo: 401.1589 Found: 401.1556 Rt: 2.25 min |

The following compounds were similarly prepared by a method analogous to that described for Example 1, but isolated as the free base.

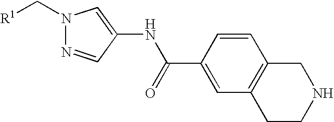

TABLE 9

| Example No. | R¹ | From | Physical data |
|---|---|---|---|
| 7 N-{1-[(3- chlorophenyl)methyl]-1H- pyrazol-4-yl}-1,2,3,4- tetrahydro-6- isoquinolinecarboxamide | 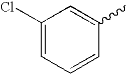 | 1,1-dimethylethyl 6- [({1-[(3-chlorophenyl) methyl]-1H-pyrazol-4- yl}amino)carbonyl]-3,4- dihydro-2(1H)- isoquinolinecarboxylate (Intermediate 16) | HRMS (M + H)⁺ calculated for C₂₀H₁₉ClN₄O Theo: 367.85 Found: 367.17 Rt: 2.24 min |
| 8 N-{1-[(2- chlorophenyl)methyl]-1H- pyrazol-4-yl}-1,2,3,4- tetrahydro-6- isoquinolinecarboxamide | 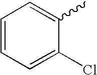 | 1,1-dimethylethyl 6- [({1-[(2-chlorophenyl) methyl]-1H-pyrazol-4- yl}amino)carbonyl]-3,4- dihydro-2(1H)- isoquinolinecarboxylate (Intermediate 17) | HRMS (M + H)⁺ calculated for C₂₀H₁₉ClN₄O Theo: 367.85 Found: 367.14 Rt: 2.24 min |
| 9 N-(1-{[3- (methyloxy)phenyl]methyl}- 1H-pyrazol-4-yl)-1,2,3,4- tetrahydro-6- isoquinolinecarboxamide | 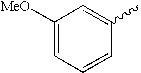 | 1,1-dimethylethyl 6-{[(1-{[3-(methyloxy) phenyl]methyl}-1H- pyrazol-4-yl) amino]carbonyl}-3,4- dihydro-2(1H)- isoquinolinecarboxylate (Intermediate 18) | HRMS (M + H)⁺ calculated for C₂₁H₂₂N₄O₂ Theo: 363.43 Found: 363.18 Rt: 2.20 min |
| 10 N-{1-[(3- fluorophenyl)methyl]-1H- pyrazol-4-yl}-1,2,3,4- tetrahydro-6- isoquinolinecarboxamide | 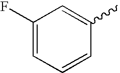 | 1,1-dimethylethyl 6- [({1-[(3-fluorophenyl) methyl]-1H-pyrazol-4- yl}amino)carbonyl]-3,4- dihydro-2(1H)- isoquinolinecarboxylate (Intermediate 19) | HRMS (M + H)⁺ calculated for C₂₀H₁₉FN₄O Theo: 351.39 Found: 351.23 Rt: 2.21 min |
| 11 N-(1-{[3- (trifluoromethyl)phenyl]meth- yl}-1H-pyrazol-4-yl)- 1,2,3,4-tetrahydro-6- isoquinolinecarboxamide | 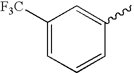 | 1,1-dimethylethyl 6- {[(1-{[3-(trifluoromethyl) phenyl]methyl}-1H- pyrazol-4- yl)amino]carbonyl}-3,4- dihydro-2(1H)- isoquinolinecarboxylate (Intermediate 20) | HRMS (M + H)⁺ calculated for C₂₁H₁₉F₃N₄O Theo: 401.40 Found: 401.19 Rt: 2.44 min |
| 12 N-{1-[(4- chlorophenyl)methyl]-1H- pyrazol-4-yl}-1,2,3,4- tetrahydro-6- isoquinolinecarboxamide | 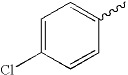 | 1,1-dimethylethyl 6- [({1-[(4-chlorophenyl) methyl]-1H-pyrazol-4- yl}amino)carbonyl]-3,4- dihydro-2(1H)- isoquinolinecarboxylate (Intermediate 21) | HRMS (M + H)⁺ calculated for C₂₀H₁₉ClN₄O Theo: 367.85 Found: 367.25 Rt: 2.28 min |
| 13 N-{1-[(3- methylphenyl)methyl]-1H- pyrazol-4-yl}-1,2,3,4- tetrahydro-6- isoquinolinecarboxamide | 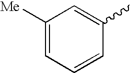 | 1,1-dimethylethyl 6- [({1-[(3- methylphenyl)methyl]- 1H-pyrazol-4- yl}amino)carbonyl]-3,4- dihydro-2(1H)- isoquinolinecarboxylate (Intermediate 22) | HRMS (M + H)⁺ calculated for C₂₁H₂₂N₄O Theo: 347.43 Found: 347.37 Rt: 2.22 min |
| 14 N-{1-[(4- methylphenyl)methyl]-1H- pyrazol-4-yl}-1,2,3,4- tetrahydro-6- isoquinolinecarboxamide | 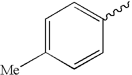 | 1,1-dimethylethyl 6- [({1-[(4- methylphenyl)methyl]- 1H-pyrazol-4- yl}amino)carbonyl]-3,4- dihydro-2(1H)- isoquinolinecarboxylate (Intermediate 23) | HRMS (M + H)⁺ calculated for C₂₁H₂₂N₄O Theo: 347.43 Found: 347.31 Rt: 2.23 min |
| 15 N-{1-[(4- fluorophenyl)methyl]-1H- pyrazol-4-yl}-1,2,3,4- tetrahydro-6- isoquinolinecarboxamide | 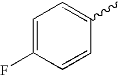 | 1,1-dimethylethyl 6- [({1-[(4- fluorophenyl)methyl]- 1H-pyrazol-4- yl}amino)carbonyl]-3,4- dihydro-2(1H)- isoquinolinecarboxylate (Intermediate 24) | HRMS (M + H)⁺ calculated for C₂₀H₁₉FN₄O Theo: 351.39 Found: 351.16 Rt: 2.22 min |

### EXAMPLE 16

### 1-[(3,4-Dichlorophenyl)methyl]-N-(1,2,3,4-tetrahydro-6-isoquinolinyl)-1H-pyrazole-4-carboxamide hydrochloride

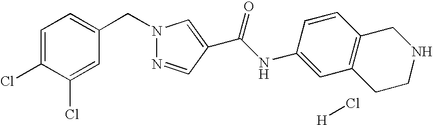

To a solution of 1,1-Dimethylethyl 6-[({1-[(3,4-dichlorophenyl)methyl]-1H-pyrazol-4-yl}carbonyl)amino]-3,4-dihydro-2(1H)-isoquinolinecarboxylate (Intermediate 28) (0.12 g, 0.24 mmol) in EtOAc (5 mL) was bubbled HCl (g) and the reaction mixture was stirred at room temperature overnight. The resulting precipitate was filtered and recrystallized from EtOH to give the title compound as a white solid (35 mg, 36%).

HRMS calculated for C₂₀H₁₈Cl₂N₄O (M+H)⁺ 401.0936, found: 401.0939, Rt: 2.26 min.

The following compounds were similarly prepared by a method analogous to that described for Example 16.

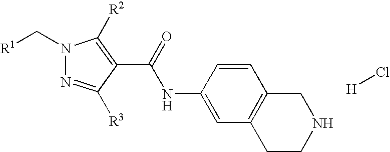

TABLE 10

| Example No. | R¹ | R² | R³ | From | Physical data |
|---|---|---|---|---|---|
| 17 1-(phenylmethyl)-N- (1,2,3,4-tetrahydro-6- isoquinolinyl)-1H- pyrazole-4-carboxamide hydrochloride | 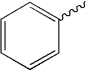 | H | H | 1,1-dimethylethyl 6-({[1- (phenylmethyl)-1H- pyrazol-4- yl]carbonyl}amino)-3,4- dihydro-2(1H)- isoquinolinecarboxylate (Intermediate 29) | HRMS (M + H)⁺ calculated for C₂₀H₂₀N₄O Theo: 333.1715 Found: 333.1702 Rt: 2.08 min |
| 18 1-[(3,4- dichlorophenyl)methyl]- 5-methyl-N-(1,2,3,4- tetrahydro-6- isoquinolinyl)-1H- pyrazole-4-carboxamide hydrochloride | 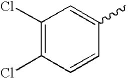 | Me | H | 1,1-dimethylethyl 6-[({1- [(3,4- dichlorophenyl)methyl]- 5-methyl-1H-pyrazol-4- yl}carbonyl)amino]-3,4- dihydro-2(1H)- isoquinolinecarboxylate (Intermediate 30) | HRMS (M + H)⁺ calculated for C₂₁H₂₀Cl₂N₄O Theo: 415.1092 Found: 415.1068 Rt: 2.58 min |
| 19 1-[(3,4- dichlorophenyl)methyl]- 3-methyl-N-(1,2,3,4- tetrahydro-6- isoquinolinyl)-1H- pyrazole-4-carboxamide hydrochloride | 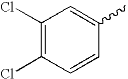 | H | Me | 1,1-dimethylethyl 6-[({1- [(3,4- dichlorophenyl)methyl]- 3-methyl-1H-pyrazol-4- yl}carbonyl)amino]-3,4- dihydro-2(1H)- isoquinolinecarboxylate (Intermediate 31) | HRMS (M + H)⁺ calculated for C₂₁H₂₀Cl₂N₄O Theo: 415.1092 Found: 415.1105 Rt: 2.54 min |
| 20 N-(1,2,3,4-tetrahydro-6- isoquinolinyl)-1-{[3- (trifluoromethyl)phenyl]meth- yl}-1H-pyrazole-4- carboxamide | 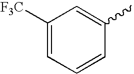 | H | H | 1,1-dimethylethyl 6-{[(1- {[3- (trifluoromethyl)phenyl] methyl}-1H-pyrazol-4- yl)carbonyl]amino}-3,4- dihydro-2(1H)- isoquinolinecarboxylate (Intermediate 32) | HRMS (M + H)⁺ calculated for C₂₁H₁₉F₃N₄O Theo: 401.1589 Found: 401.1620 Rt: 2.31 min |
| 21 1-(1- naphthalenylmethyl)-N- (1,2,3,4-tetrahydro-6- isoquinolinyl)-1H- pyrazole-4-carboxamide | 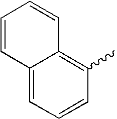 | H | H | 1,1-dimethylethyl 6-({[1- (1-naphthalenylmethyl)- 1H-pyrazol-4- yl]carbonyl}amino)-3,4- dihydro-2(1H)- isoquinolinecarboxylate (Intermediate 33) | HRMS (M + H)⁺ calculated for C₂₄H₂₂N₄O Theo: 383.1872 Found: 383.1847 Rt: 2.22 min |
| 22 1-[(4- fluorophenyl)methyl]-N- (1,2,3,4-tetrahydro-6- isoquinolinyl)-1H- pyrazole-4-carboxamide | 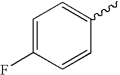 | H | H | 1,1-dimethylethyl 6-[({1- [(4- fluorophenyl)methyl]- 1H-pyrazol-4- yl}carbonyl)amino]-3,4- dihydro-2(1H)- isoquinolinecarboxylate (Intermediate 35) | HRMS (M + H)⁺ calculated for C₂₀H₁₉FN₄O Theo: 351.1621 Found: 351.1640 Rt: 1.97 min |

### EXAMPLE 23

### N-{1-[(3,4-Dichlorophenyl)methyl]-1H-pyrazol-4-yl}-2-propyl-1,2,3,4-tetrahydro-6-isoquinolinecarboxamide hydrochloride

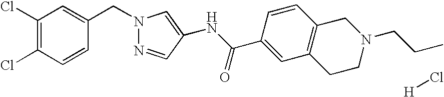

To a solution of 2-propyl-1,2,3,4-tetrahydro-6-isoquinolinecarboxylic acid (50 mg, 0.19 mmol), N-(3-dimethylaminopropyl)-N′-ethylcarbodiimide hydrochloride (45 mg, 0.23 mmol), 1-hydroxybenzotriazole hydrate (32 mg, 0.23 mmol) and triethylamine (63 mg, 0.62 mmol) in DCM (5 mL) was added 1-[(3,4-dichlorophenyl)methyl]-1H-pyrazol-4-amine (Intermediate 7) as the hydrochloride salt (54 mg, 0.19 mmol) and the reaction mixture was stirred at room temperature overnight. The organic phase was then washed with a 1N sodium hydroxide solution, with brine, dried over Na₂SO₄, filtered and evaporated under reduced pressure. The residue was purified by flash column chromatography eluting with DCM/MeOH:92/8 and the oily residue was dissolved in EtOAc and a 1N solution of HCl in diethyl ether was added. The precipitate was then filtered and recrystallized from CH₃CN to give the title compound as a pale yellow solid (35 mg, 38%).

HRMS calculated for C₂₃H₂₄Cl₂N₄O (M+H)⁺ 443.1405, found: 443.1401, Rt: 2.93 min.

### EXAMPLE 24

### 1,1-Dimethylethyl 6-[({1-[(3,4-dichlorophenyl)methyl]-1H-pyrazol-4-yl}amino)carbonyl]-3,4-dihydro-2(1H)-isoquinolinecarboxylate

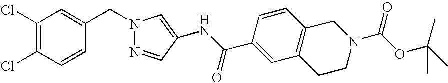

To a solution of 2-{[(1,1-dimethylethyl)oxy]carbonyl}-1,2,3,4-tetrahydro-6-isoquinoline carboxylic acid (138 mg, 0.5 mmol), N-(3-dimethylaminopropyl)-N′-ethylcarbodiimide hydrochloride (114 mg, 0.6 mmol), 1-hydroxybenzotriazole hydrate (81 mg, 0.6 mmol) and triethylamine (60 mg, 0.6 mmol) in DCM (10 mL) was added 1-[(3,4-dichlorophenyl)methyl]-1H-pyrazol-4-amine (Intermediate 7) (120 mg, 0.5 mmol) and the reaction mixture was stirred at room temperature overnight. The organic phase was then washed with a 1N sodium hydroxide solution, with brine, dried over Na₂SO₄, filtered and evaporated under reduced pressure. The residue was purified by flash column chromatography eluting with DCM/MeOH:95/5 to give the title compound as a white solid (190 mg, 77%) after recrystallization from CH₃CN.

LC/MS: m/z 501 (M+H)⁺, Rt: 3.65 min.

### EXAMPLE 25

### 1,1-dimethylethyl 6-[({1-[(3-cyanophenyl)methyl]-1H-pyrazol-4-yl}amino)carbonyl]-3,4-dihydro-2(1H)-isoquinolinecarboxylate

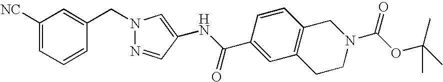

To a mixture of 1,1-dimethylethyl 6-[(1H-pyrazol-4-ylamino)carbonyl]-3,4-dihydro-2(1H)-isoquinolinecarboxylate (Intermediate 25) (150 mg, 0.44 mmol) and cesium carbonate (171 mg, 0.53 mmol) in DMF was added 3-(bromomethyl)benzonitrile (172 mg, 0.88 mmol). The reaction mixture was stirred at 80° C. for 2 days. The solvent was then evaporated under reduced pressure and the residue was put in dichloromethane. The organic phase was washed with brine, dried over Na₂SO₄, filtered and evaporated under reduced pressure. The residue was purified by flash column chromatography eluting with a gradient DCM 100% to DCM/MeOH:94/6 to give after trituration in pentane the title compound as an off-white solid (100 mg, 50%).

HRMS calculated for C₂₆H₂₇N₅O₃ (M+H)⁺ 458.2192, found: 458.2212, Rt: 2.88 min

### EXAMPLE 26

### 2-Acetyl-N-{1-[(3,4-dichlorophenyl)methyl]-1H-pyrazol-4-yl}-1,2,3,4-tetrahydro-6-isoquinolinecarboxamide

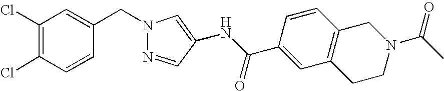

To a solution of N-{1-[(3,4-dichlorophenyl)methyl]-1H-pyrazol-4-yl}-1,2,3,4-tetrahydro-6-isoquinolinecarboxamide hydrochloride (Example 1) (80 mg, 0.18 mmol) in THF (5 mL) were added pyridine (45 μL, 0.56 mmol) and acetyl chloride (15 μL, 0.21 mmol). The reaction mixture was stirred at room temperature for 4 hours. After a reaction check, an extra amount of acetyl chloride (1 drop) was added and the reaction mixture was stirred overnight. Dichloromethane was added and the organic layer was washed with 1N hydrochloride acid then 1N sodium hydroxide aqueous solution. The organic phase was dried over sodium sulphate. After filtration and evaporation under reduced pressure, the residue was purified by flash column chromatography eluting with a gradient DCM 100% to DCM/MeOH:92/8 to give the title compound as an oil which crystallised to a white solid (15 mg, 19%) after trituration with pentane and dichloromethane.

HRMS calculated for C₂₂H₂₀Cl₂N₄O₂ (M+H)⁺ 443.1042, found: 443.1039, Rt: 2.55 min

The following compounds were similarly prepared by analogous method to that described for Example 26.

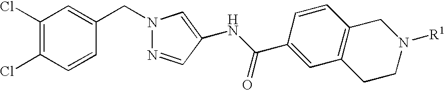

TABLE 11

| Example No | R⁴ | From | Physical data |
|---|---|---|---|
| 27 2-butanoyl-N-{1-[(3,4- dichlorophenyl)methyl]-1H- pyrazol-4-yl}-1,2,3,4- tetrahydro-6- isoquinolinecarboxamide | 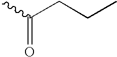 | N-{1-[(3,4- Dichlorophenyl)methyl]- 1H-pyrazol-4-yl}-1,2,3,4- tetrahydro-6- isoquinolinecarboxamide hydrochloride (Example 1) | HRMS (M + H)⁺ calculated for C₂₄H₂₄Cl₂N₄O₂ Theo: 471.1354 Found: 471.1360 Rt: 2.85 min |
| 28 2-(cyclopentylcarbonyl)-N- {1-[(3,4- dichlorophenyl)methyl]-1H- pyrazol-4-yl}-1,2,3,4- tetrahydro-6- isoquinolinecarboxamide | 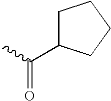 | N-{1-[(3,4- Dichlorophenyl)methyl]- 1H-pyrazol-4-yl}-1,2,3,4- tetrahydro-6- isoquinolinecarboxamide hydrochloride (Example 1) | HRMS (M + H)⁺ calculated for C₂₆H₂₆Cl₂N₄O₂ Theo: 497.1511 Found: 497.1276 Rt: 3.02 min |

### EXAMPLE 29

### N-{1-[(3,4-dichlorophenyl)methyl]-1H-pyrazol-4-yl}-2-(4-methylpentanoyl)-1,2,3,4-tetrahydro-6-isoquinolinecarboxamide

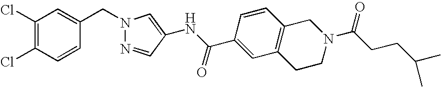

A solution of N-{1-[(3,4-dichlorophenyl)methyl]-1H-pyrazol-4-yl}-1,2,3,4-tetrahydro-6-isoquinolinecarboxamide hydrochloride (Example 1) (105 mg, 0.24 mmol), 4-methylpentanoic acid (40 μL, 0.32 mmol), HOBt (40 mg, 0.29 mmol), EDCl (55 mg, 0.29 mmol) and triethylamine (100 μL, 1.3 mmol) in dichloromethane (5 mL) was stirred at room temperature for 4 days. The reaction mixture was washed with a 1N sodium hydroxide aqueous solution then dried over sodium sulphate. After filtration and evaporation under reduced pressure, the residue was purified by flash column chromatography eluting with a gradient DCM 100% to DCM/MeOH:94/6 to give the title compound as colourless oil (75 mg, 63%).

HRMS calculated for C₂₆H₂₈Cl₂N₄O₂ (M+H)⁺ 499.1667, found: 499.1659, Rt: 3.12 min

The following compounds were similarly prepared by a method analogous to that described for Example 1.

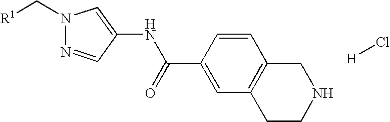

TABLE 12

| Example No. | R¹ | From | Physical data |
|---|---|---|---|
| 30 N-(1-{[(3,4- dichlorophenyl)oxy]methyl}- 1H-pyrazol-4-yl)-1,2,3,4- tetrahydro-6- isoquinolinecarboxamide hydrochloride | 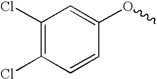 | 1,1-dimethylethyl 6-{[(1- {[(3,4- dichlorophenyl)oxy]methyl}- 1H-pyrazol-4- yl)amino]carbonyl}-3,4- dihydro-2(1H)- isoquinolinecarboxylate (intermediate 36) | HRMS (M + H)⁺ calculated for C₂₀H₁₈Cl₂N₄O₂ Theo: 417.0885 Found: 417.0905 Rt: 2.40 min |
| 31 N-[1-({[3- (trifluoromethyl)phenyl]oxy} methyl)-1H-pyrazol-4- yl]-1,2,3,4-tetrahydro-6- isoquinolinecarboxamide hydrochloride | 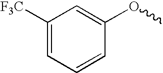 | 1,1-dimethylethyl 6-({[1-({[3- (trifluoromethyl)phenyl]oxy} methyl)-1H-pyrazol-4- yl]amino}carbonyl)-3,4- dihydro-2(1H)- isoquinolinecarboxylate (intermediate 37) | HRMS (M + H)⁺ calculated for C₂₁H₁₉F₃N₄O₂ Theo: 417.1538 Found: 417.1530 Rt: 2.35 min |
| 32 N-(1-{[(2,5- dichlorophenyl)oxy]methyl}- 1H-pyrazol-4-yl)-1,2,3,4- tetrahydro-6- isoquinolinecarboxamide hydrochloride | 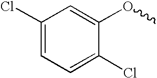 | 1,1-dimethylethyl 6-{[(1- {[(2,5- dichlorophenyl)oxy]methyl}- 1H-pyrazol-4- yl)amino]carbonyl}-3,4- dihydro-2(1H)- isoquinolinecarboxylate (intermediate 38) | HRMS (M + H)⁺ calculated for C₂₀H₁₈Cl₂N₄O₂ Theo: 417.0885 Found: 417.0911 Rt: 2.37 min |
| 33 N-(1-{[(2,4- dichlorophenyl)oxy]methyl}- 1H-pyrazol-4-yl)-1,2,3,4- tetrahydro-6- isoquinolinecarboxamide hydrochloride | 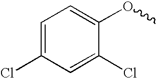 | 1,1-dimethylethyl 6-{[(1- {[(2,4- dichlorophenyl)oxy]methyl}- 1H-pyrazol-4- yl)amino]carbonyl}-3,4- dihydro-2(1H)- isoquinolinecarboxylate (intermediate 39) | HRMS (M + H)⁺ calculated for C₂₀H₁₈Cl₂N₄O₂ Theo: 417.0885 Found: 417.0876 Rt: 2.38 min |
| 34 N-(1-{[(3- chlorophenyl)oxy]methyl}- 1H-pyrazol-4-yl)-1,2,3,4- tetrahydro-6- isoquinolinecarboxamide hydrochloride | 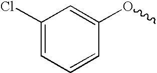 | 1,1-dimethylethyl 6-{[(1-{[(3- chlorophenyl)oxy]methyl}- 1H-pyrazol-4- yl)amino]carbonyl}-3,4- dihydro-2(1H)- isoquinolinecarboxylate (intermediate 40) | HRMS (M + H)⁺ calculated for C₂₀H₁₉ClN₄O₂ Theo: 383.1275 Found: 383.1255 Rt: 2.23 min |

### Biological Assay

The compounds of the present invention may be analysed in vitro for SCD activity using an assay based on the production of [³H]H₂O, which is released during the enzyme-catalyzed generation of the monounsaturated fatty acyl CoA product. The assay is performed in a 96-well filtration plates. The titrated substrate used in the assay is the [9,10-³H] stearoyl Coenzyme A. After incubation for 6 minutes of SCD-containing rat microsomes (2 μg protein) and substrate (1 μM), the labelled fatty acid acyl-CoA species and microsomes are absorbed with charcoal and separated from [³H]H₂O by centrifugation. The formation of [³H]H₂O is used as a measure of SCD activity. Compounds at concentrations starting at 10 μM to 0.1 nM or vehicle (DMSO) are preincubated for 5 minutes with the microsomes before addition of the substrate. The concentration-responses are fitted with sigmoidal curves to obtain IC₅₀ values. All of the synthetic Example compounds tested by the above described in vitro assay for SCD activity were found to exhibit an average pIC₅₀ value of greater than 5.

All of the synthetic Example compounds 1 to 7, 9 to 13 and 15 to 29 tested by the above described in vitro assay for SCD activity were found to exhibit an average pIC₅₀ value of greater than 5.5.

The following compounds were prepared according similar protocols to above described and when tested by the above described in vitro assay for SCD activity were found to exhibit an average pIC₅₀ value in the range 5-5.5.

| Structure | Name |
|---|---|
| 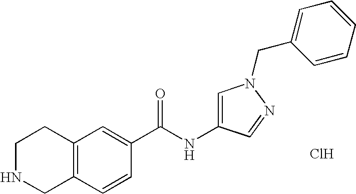 | N-[1-(phenylmethyl)-1H-pyrazol-4- yl]-1,2,3,4-tetrahydro-6- isoquinolinecarboxamide hydrochloride |
| 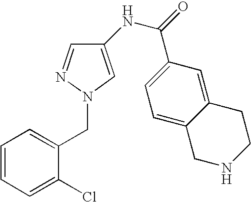 | Example 8 N-{1-[(2-chlorophenyl)methyl]-1H- pyrazol-4-yl}-1,2,3,4-tetrahydro-6- isoquinolinecarboxamide |
| 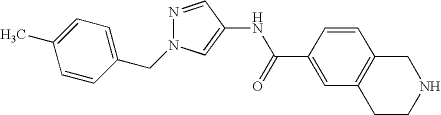 | Example 14 N-{1-[(4-methylphenyl)methyl]-1H- pyrazol-4-yl}-1,2,3,4-tetrahydro-6- isoquinolinecarboxamide |
| 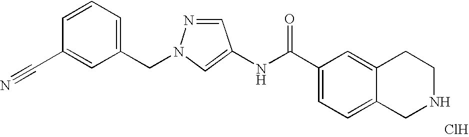 | N-{1-[(3-cyanophenyl)methyl]-1H- pyrazol-4-yl}-1,2,3,4-tetrahydro-6- isoquinolinecarboxamide hydrochloride |
| 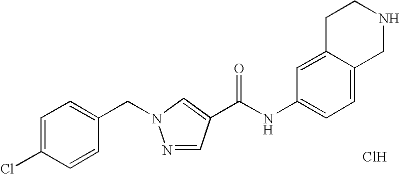 | 1-[(4-chlorophenyl)methyl]-N- (1,2,3,4-tetrahydro-6-isoquinolinyl)- 1H-pyrazole-4-carboxamide hydrochloride |
| 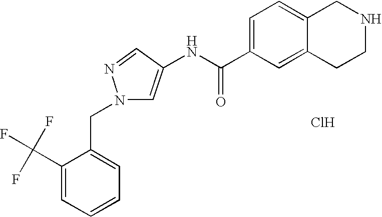 | N-(1-{[2- (trifluoromethyl)phenyl]methyl}-1H- pyrazol-4-yl)-1,2,3,4-tetrahydro-6- isoquinolinecarboxamide hydrochloride |
| 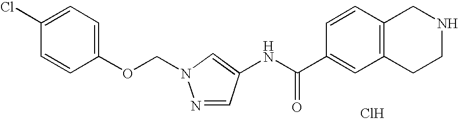 | N-(1-{[(4-chlorophenyl)oxy]methyl}- 1H-pyrazol-4-yl)-1,2,3,4-tetrahydro- 6-isoquinolinecarboxamide hydrochloride |

The following compounds were also prepared and when tested by the above described in vitro assay for SCD activity were found to exhibit an average pIC₅₀ value of less than 5.

| Structure | Name |
|---|---|
| 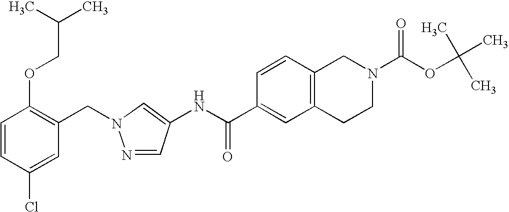 | 1,1-dimethylethyl 6-({[1-({5-chloro-2- [(2- methylpropyl)oxy]phenyl}methyl)- 1H-pyrazol-4-yl]amino}carbonyl)- 3,4-dihydro-2(1H)- isoquinolinecarboxylate |
| 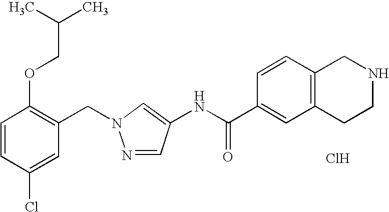 | N-[1-({5-chloro-2-[(2- methylpropyl)oxy]phenyl}methyl)- 1H-pyrazol-4-yl]-1,2,3,4-tetrahydro- 6-isoquinolinecarboxamide hydrochloride |
| 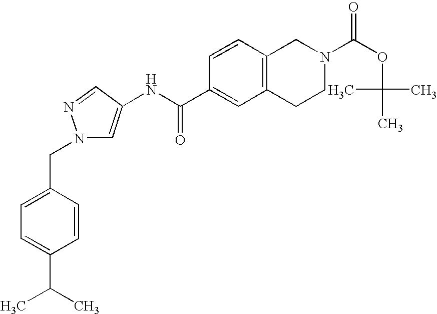 | 1,1-dimethylethyl 6-{[(1-{[4-(1- methylethyl)phenyl]methyl}-1H- pyrazol-4-yl)amino]carbonyl}-3,4- dihydro-2(1H)- isoquinolinecarboxylate |
| 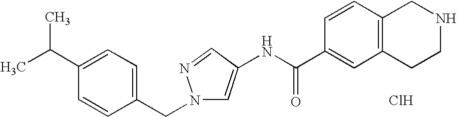 | N-(1-{[4-(1- methylethyl)phenyl]methyl}-1H- pyrazol-4-yl)-1,2,3,4-tetrahydro-6- isoquinolinecarboxamide hydrochloride |
| 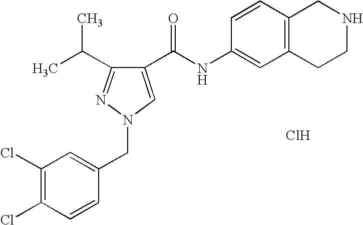 | 1-[(3,4-dichlorophenyl)methyl]-3-(1- methylethyl)-N-(1,2,3,4-tetrahydro- 6-isoquinolinyl)-1H-pyrazole-4- carboxamide hydrochloride |
| 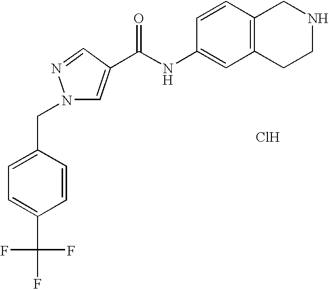 | N-(1,2,3,4-tetrahydro-6- isoquinolinyl)-1-{[4- (trifluoromethyl)phenyl]methyl}-1H- pyrazole-4-carboxamide hydrochloride |
| 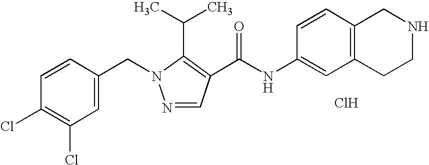 | 1-[(3,4-dichlorophenyl)methyl]-5-(1- methylethyl)-N-(1,2,3,4-tetrahydro- 6-isoquinolinyl)-1H-pyrazole-4- carboxamide hydrochloride |

1. A compound of formula (I): 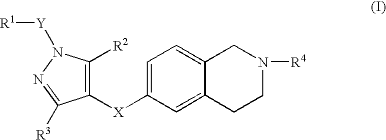
wherein:
X represents —CONH—, —NHCO— or —NHCONH—,
R¹ represents:
(i) H;
(ii) —C₁₋₆alkyl;
(iii) —C₃₋₆cycloalkyl;
(iv) —C₆₋₁₀aryl optionally substituted by one, two or three groups independently selected from:
(—C₁₋₆alkyl, —C₁₋₆haloalkyl, —C₃₋₆cycloalkyl, —C₁₋₆alkoxy, —OR⁵, —CN, —CN, halogen,
—C₆₋₁₀aryl, —C₅₋₁₀heteroaryl and —C₅₋₁₀heterocyclyl, wherein the —C₆₋₁₀aryl, —C₅₋₁₀heteroaryl and —C₅₋₁₀heterocyclyl ring is optionally substituted by one, two or three groups independently selected from: —C₁₋₆alkyl, —OR⁵, —C₁₋₆alkoxy, —C₁₋₆haloalkyl, —CN and halogen;
(v) —C₅₋₁₀heteroaryl optionally substituted by one, two or three groups independently selected from:
—C₁₋₆alkyl, —C₁₋₆haloalkyl, —C₃₋₆cycloalkyl, —C₁₋₆alkoxy, —OR⁵, —CN, halogen,
—C₆₋₁₀aryl, —C₅₋₁₀heteroaryl and —C₅₋₁₀heterocyclyl wherein the —C₆₋₁₀aryl, —C₅₋₁₀heteroaryl and —C₅₋₁₀heterocyclyl ring is optionally substituted by one, two or three groups independently selected from: —C₁₋₆alkyl, —OR⁵, —C₁₋₆alkoxy, —C₁₋₆haloalkyl, —CN and halogen; or
(vi) C₅₋₁₀heterocyclyl optionally substituted by one, two or three groups independently selected from: —C₁₋₆alkyl, —C₁₋₆alkoxy, —OR⁵, —CN, halogen, —C₆₋₁₀aryl, —C₅₋₁₀heteroaryl and —C₅₋₁₀heterocyclyl wherein the —C₆₋₁₀aryl, —C₅₋₁₀heteroaryl and —C₅₋₁₀heterocyclyl ring is optionally substituted by one, two or three groups independently selected from: —C₁₋₆alkyl, —OR⁵, —C₁₋₆alkoxy, —C₁₋₆haloalkyl, —CN and halogen,
Y represents —(CH₂)ₘ— or —O(CH₂)ₘ—,
one of R² and R³ represents hydrogen and the other represents H, —C₁₋₆alkyl or —C₃₋₆ cycloalkyl,
R⁴ represents H, —C₁₋₆alkyl, —C(═O)C₁₋₆alkyl, —C(═O)C₃₋₆cycloalkyl or —CO₂C₁₋₆alkyl,
R⁵ represents —C₁₋₆haloalkyl or —C₃₋₆cycloalkyl, and
m represents 1-3,
or a pharmaceutically acceptable salt thereof.

2. The compound of formula (I) or pharmaceutically acceptable salt thereof according to claim 1 wherein X represents —NHCO—.

3. The compound of formula (I) or pharmaceutically acceptable salt thereof according to claim 1 wherein Y represents —CH₂—.

4. The compound of formula (I) or pharmaceutically acceptable salt thereof according to claim 1 wherein R¹ represents —C₆₋₁₀aryl optionally substituted by one, two or three groups independently selected from:
—C₁₋₆alkyl, —C₁₋₆haloalkyl, —C₃₋₆cycloalkyl, —C₁₋₆alkoxy, —OR⁵, —CN, halogen, and
—C₆₋₁₀aryl optionally substituted by one, two or three groups selected from: —C₁₋₆alkyl, —OR⁵, —C₁₋₆alkoxy, —C₁₋₆haloalkyl, —CN of and halogen.

5. The compound of formula (I) or pharmaceutically acceptable salt thereof according to claim 1 wherein R² represents hydrogen.

6. The compound of formula (I) or pharmaceutically acceptable salt thereof according to claim 1 wherein R³ represents hydrogen.

7. The compound of formula (I) or pharmaceutically acceptable salt thereof according to claim 1 wherein R⁴ represents hydrogen.

8. The compound of formula (I) or a pharmaceutically acceptable salt thereof according to claim 1 wherein the compound of formula (I) is selected from:
N-{1-[(3,4-Dichlorophenyl)methyl]-1H-pyrazol-4-yl}-1,2,3,4-tetrahydro-6-isoquinolinecarboxamide,
N-[1-(1-naphthalenylmethyl)-1H-pyrazol-4-yl]-1,2,3,4-tetrahydro-6-isoquinolinecarboxamide,
N-{1-[(2,4-dichlorophenyl)methyl]-1H-pyrazol-4-yl}-1,2,3,4-tetrahydro-6-isoquinolinecarboxamide,
N-{1-[(2-methylphenyl)methyl]-1H-pyrazol-4-yl}-1,2,3,4-tetrahydro-6-isoquinolinecarboxamide,
N-[1-(cyclohexylmethyl)-1H-pyrazol-4-yl]-1,2,3,4-tetrahydro-6-isoquinolinecarboxamide,
N-(1-{[4-(trifluoromethyl)phenyl]methyl}-1H-pyrazol-4-yl)-1,2,3,4-tetrahydro-6-isoquinolinecarboxamide,
N-{1-[(3-chlorophenyl)methyl]-1H-pyrazol-4-yl}-1,2,3,4-tetrahydro-6-isoquinolinecarboxamide,
N-{1-[(2-chlorophenyl)methyl]-1H-pyrazol-4-yl}-1,2,3,4-tetrahydro-6-isoquinolinecarboxamide,
N-(1-{[3-(methyloxy)phenyl]methyl}1H-pyrazol-4-yl)-1,2,3,4-tetrahydro-6-isoquinolinecarboxamide,
N-{1-[(3-fluorophenyl)methyl]-1H-pyrazol-4-yl}-1,2,3,4-tetrahydro-6-isoquinolinecarboxamide,
N-(1-{[3-(trifluoromethyl)phenyl]methyl}-1H-pyrazol-4-yl)-1,2,3,4-tetrahydro-6-isoquinolinecarboxamide,
N-{1-[(4-chlorophenyl)methyl]-1H-pyrazol-4-yl}-1,2,3,4-tetrahydro-6-isoquinolinecarboxamide,
N-{1-[(3-methylphenyl)methyl]-1H-pyrazol-4-yl}-1,2,3,4-tetrahydro-6-isoquinolinecarboxamide,
N-{1-[(4-methylphenyl)methyl]-1H-pyrazol-4-yl}-1,2,3,4-tetrahydro-6-isoquinolinecarboxamide,
N-{1-[(4-fluorophenyl)methyl]-1H-pyrazol-4-yl}-1,2,3,4-tetrahydro-6-isoquinolinecarboxamide,
1-[(3,4-Dichlorophenyl)methyl]-N-(1,2,3,4-tetrahydro-6-isoquinolinyl)-1H-pyrazole-4-carboxamide hydrochloride,
1-(phenylmethyl)-N-(1,2,3,4-tetrahydro-6-isoquinolinyl)-1H-pyrazole-4-carboxamide hydrochloride,
1-[(3,4-dichlorophenyl)methyl]-5-methyl-N-(1,2,3,4-tetrahydro-6-isoquinolinyl)-1H-pyrazole-4-carboxamide,
1-[(3,4-dichlorophenyl)methyl]-3-methyl-N-(1,2,3,4-tetrahydro-6-isoquinolinyl)-1H-pyrazole-4-carboxamide hydrochloride,
N-(1,2,3,4-tetrahydro-6-isoquinolinyl)-1-{[3-(trifluoromethyl)phenyl]methyl}-1H-pyrazole-4-carboxamide,
1-(1-naphthalenylmethyl)-N-(1,2,3,4-tetrahydro-6-isoquinolinyl)-1H-pyrazole-4-carboxamide,
1-[(4-fluorophenyl)methyl]-N-(1,2,3,4-tetrahydro-6-isoquinolinyl)-1H-pyrazole-4-carboxamide,
N-{1-[(3,4-dichlorophenyl)methyl]-1H-pyrazol-4-yl}-2-propyl-1,2,3,4-tetrahydro-6-isoquinolinecarboxamide,
1,1-Dimethylethyl 6-[({1-[(3,4-dichlorophenyl)methyl]-1H-pyrazol-4-yl}amino)carbonyl]-3,4-dihydro-2(1H)-isoquinolinecarboxylate,
1,1-dimethylethyl 6-[({1-[(3-cyanophenyl)methyl]-1H-pyrazol-4-yl}amino)carbonyl]-3,4-dihydro-2(1H)-isoquinoinecarboxylate,
2-acetyl-N-{1-[(3,4-dichlorophenyl)methyl]-1H-pyrazol-4-yl}-1,2,3,4-tetrahydro-6-isoquinolinecarboxamide,
2-butanoyl-N-{1-[(3,4-dichlorophenyl)methyl]-1H-pyrazol-4-yl}-1,2,3,4-tetrahydro-6-isoquinolinecarboxamide,
2-(cyclopentylcarbonyl)-N-{1-[(3,4-dichlorophenyl)methyl]-1H-pyrazol-4-yl}-1,2,3,4-tetrahydro-6-isoquinolinecarboxamide,
N-{1-[(3,4-dichlorophenyl)methyl]-1H-pyrazol-4-yl}-2-(4-methylpentanoyl)-1,2,3,4-tetrahydro-6-isoquinolinecarboxamide,
N-(1-{[(3,4-dichlorophenyl)oxy]methyl}-1H-pyrazol-4-yl)-1,2,3,4-tetrahydro-6-isoquinolinecarboxamide hydrochloride,
N-[1-({[3-(trifluoromethyl)phenyl]oxy}methyl)-1H-pyrazol-4-yl]-1,2,3,4-tetrahydro-6-isoquinolinecarboxamide,
N-(1-{[(2,5-dichlorophenyl)oxy]methyl}-1H-pyrazol-4-yl)-1,2,3,4-tetrahydro-6-isoquinolinecarboxamide,
N-(1-{[(2,4-dichlorophenyl)oxy]methyl}-1H-pyrazol-4-yl)-1,2,3,4-tetrahydro-6-isoquinolinecarboxamide, and
N-(1-({[3-chlorophenyl)oxy]methyl}-1H-pyrazol-4-yl)-1,2,3,4-tetrahydro-6-isoquinolinecarboxamide.

9. A pharmaceutical composition comprising the compound of formula (I) or pharmaceutically acceptable salt thereof according to claim 1 together with at least one pharmaceutical carrier and/or excipient.

10-16. (canceled)

17. A method of treating and/or preventing a disease or a condition susceptible to amelioration by an SCD inhibitor comprising administering to a subject a therapeutically effective amount of the compound of formula (I) or pharmaceutically acceptable salt thereof according to claim 1.

18. A method of treating and/or preventing diseases or conditions caused by or associated with an abnormal plasma lipid profile selected from dyslipidemia, hypoalphalipoproteinemia, hyperbetalipoproteinemia, hypercholesterolemia, hypertriglyceridemia, familial hypercholesterolemia, angina, ischemia, cardiac ischemia, stroke, myocardial infarction, atherosclerosis, obesity, Type I diabetes, Type II diabetes, insulin resistance, hyperinsulinaemia and metabolic syndrome;
cardiovascular diseases selected from peripheral vascular disease, reperfusion injury, angioplastic restenosis, hypertension, vascular complications of diabetes, and thrombosis;
hepatic steatosis, non-alcoholic steatohepatitis (NASH) or other diseases related to accumulation of lipids in the liver;
skin disorders selected from eczema, acne, psoriasis, and keloid scar formation;
diseases related to production or secretions from mucous membranes;
cancer, neoplasia, malignancy, metastases, tumours (benign or malignant), carcinogenesis, or hepatomas; or
mild cognitive impairment (MCI), Alzheimer's Disease (AD), cerebral amyloid angiopathy (CAA) or dementia associated with Down Syndrome (DS) or other neurodegenerative diseases characterized by the formation or accumulation of amyloid plaques comprising Aβ42,
comprising administering to a subject a therapeutically effective amount of the compound of formula (I) or pharmaceutically acceptable salt thereof according to claim 1.

19. A method of treating and/or preventing acne, dyslipidemia, hypertriglyceridemia, atherosclerosis, obesity, Type II diabetes, insulin resistance, hyperinsulinaemia, hepatic steatosis and/or non-alcoholic steatohepatitis (NASH) comprising administering to a subject a therapeutically effective amount of the compound of formula (I) or pharmaceutically acceptable salt thereof according to claim 1.

20. The compound of formula (I) or a pharmaceutically acceptable salt thereof according to claim 1 in combination with one or more active agent(s) selected from an inhibitor of cholesteryl ester transferase (CETP inhibitors), a HMG-CoA reductase inhibitor, a microsomal triglyceride transfer protein, a peroxisome proliferator-activated receptor activator (PPAR), a bile acid reuptake inhibitor, a cholesterol absorption inhibitor, a cholesterol synthesis inhibitor, a fibrate, niacin, an ion-exchange resin, an antioxidant, an inhibitor of AcylCoA, a AcylCoA, a cholesterol acyltransferase (ACAT inhibitor), a cannabinoid 1 antagonist, a bile acid sequestrant, a corticosteroid, a vitamin D3 derivative, a retinoid, an immunomodulator, an anti androgen, a keratolytic agent, an anti-microbial, a platinum chemotherapeutic, an antimetabolite, hydroxyurea, a taxane, a mitotic disrupter, an anthracycline, dactinomycin, an alkylating agent and a cholinesterase inhibitor; wherein the compound according to claim 1 and the one or more active agent(s) are in the same or separate formulations.

21. The method of claim 17 wherein the compound of formula (I) or a pharmaceutically acceptable salt thereof according to claim 1 is administered in combination with one or more active agent(s) selected from an inhibitor of cholesteryl ester transferase (CETP inhibitors), a HMG-CoA reductase inhibitor, a microsomal triglyceride transfer protein, a peroxisome proliferator-activated receptor activator (PPAR), a bile acid reuptake inhibitor, a cholesterol absorption inhibitor, a cholesterol synthesis inhibitor, a fibrate, niacin, an ion-exchange resin, an antioxidant, an inhibitor of AcylCoA, a cholesterol acyltransferase (ACAT inhibitor), a cannabinoid 1 antagonist, a bile acid sequestrant, a corticosteroid, a vitamin D3 derivative, a retinoid, an immunomodulator, an anti androgen, a keratolytic agent, an anti-microbial, a platinum chemotherapeutic, an antimetabolite, hydroxyurea, a taxane, a mitotic disrupter, an anthracycline, dactinomycin, an alkylating agent and a cholinesterase inhibitor.

22. The compound of formula (I) or pharmaceutically acceptable salt thereof according to claim 1 wherein:
X represents —NHCO—;
Y represents —CH₂—;
R¹ represents —C₆₋₁₀aryl optionally substituted by one, two or three groups independently selected from: —C₁₋₆alkyl, —C₁₋₆haloalkyl, —C₃₋₆cycloalkyl, —C₁₋₆alkoxy, —OR⁵, —CN, halogen, and —C₆₋₁₀aryl optionally substituted by one, two or three groups selected from: —C₁₋₆alkyl, —OR⁵, —C₁₋₆alkoxy, —C₁₋₆haloalkyl, —CN and halogen; and
R², R³ and R⁴ represent hydrogen.

23. The compound of formula (I) or pharmaceutically acceptable salt thereof according to claim 8 selected from:
1-[(3,4-Dichlorophenyl)methyl]-N-(1,2,3,4-tetrahydro-6-isoquinolinyl)-1H-pyrazole-4-carboxamide hydrochloride,
1-(phenylmethyl)-N-(1,2,3,4-tetrahydro-6-isoquinolinyl)-1H-pyrazole-4-carboxamide hydrochloride,
1-[(3,4-dichlorophenyl)methyl]-3-methyl-N-(1,2,3,4-tetrahydro-6-isoquinolinyl)-1H-pyrazole-4-carboxamide hydrochloride,
N-(1-{[(3,4-dichlorophenyl)oxy]methyl}-1H-pyrazol-4-yl)-1,2,3,4-tetrahydro-6-isoquinolinecarboxamide hydrochloride,
N-[1-({[3-(trifluoromethyl)phenyl]oxy}methyl)-1H-pyrazol-4-yl]-1,2,3,4-tetrahydro-6-isoquinolinecarboxamide hydrochloride,
N-(1-{[(2,5-dichlorophenyl)oxy]methyl}-1H-pyrazol-4-yl)-1,2,3,4-tetrahydro-6-isoquinolinecarboxamide hydrochloride,
N-(1-{[(2,4-dichlorophenyl)oxy]methyl}-1H-pyrazol-4-yl)-1,2,3,4-tetrahydro-6-isoquinolinecarboxamide hydrochloride, and
N-(1-{[(3-chlorophenyl)oxy]methyl}-1H-pyrazol-4-yl)-1,2,3,4-tetrahydro-6-isoquinolinecarboxamide hydrochloride.

24. A pharmaceutical composition comprising the compound of formula (I) or pharmaceutically acceptable salt thereof according to claim 22 together with at least one pharmaceutical carrier and/or excipient.

25. A method of treating and/or preventing acne, dyslipidemia, hypertriglyceridemia, atherosclerosis, obesity, Type II diabetes, insulin resistance, hyperinsulinaemia, hepatic steatosis and/or non-alcoholic steatohepatitis (NASH) comprising administering to a subject a therapeutically effective amount of the compound of formula (I) or pharmaceutically acceptable salt thereof according to claim 22.

26. A pharmaceutical composition comprising the compound of formula (I) or pharmaceutically acceptable salt thereof according to claim 8 together with at least one pharmaceutical carrier and/or excipient.

27. A method of treating and/or preventing acne, dyslipidemia, hypertriglyceridemia, atherosclerosis, obesity, Type II diabetes, insulin resistance, hyperinsulinaemia, hepatic steatosis and/or non-alcoholic steatohepatitis (NASH) comprising administering to a subject a therapeutically effective amount of the compound of formula (I) or pharmaceutically acceptable salt thereof according to claim 8.

28. A pharmaceutical composition comprising the compound according to claim 23 together with at least one pharmaceutical carrier and/or excipient.

29. A method of treating and/or preventing acne, dyslipidemia, hypertriglyceridemia, atherosclerosis, obesity, Type II diabetes, insulin resistance, hyperinsulinaemia, hepatic steatosis and/or non-alcoholic steatohepatitis (NASH) comprising administering to a subject a therapeutically effective amount of the compound according to claim 23.
